(19) United States
(12) Patent Application Publication
(10) Pub. No.: US 2022/0213560 A1
(43) Pub. Date: Jul. 7, 2022

(54) METHODS AND APPARATUS FOR IDENTIFYING ORGAN/TISSUE HEALTH STATUS USING TRANSCRIPTOMICS ANALYSIS OF LIQUID BIOPSY SAMPLES

(71) Applicant: Certara USA, Inc., Princeton, NJ (US)

(72) Inventors: Amin ROSTAMI-HODJEGAN, Manchester (GB); Brahim ACHOUR, Manchester (GB); Patrick SMITH, Princeton, NJ (US)

(21) Appl. No.: 17/656,299

(22) Filed: Mar. 24, 2022

Related U.S. Application Data
(63) Continuation of application No. PCT/US2020/052208, filed on Sep. 23, 2020.
(60) Provisional application No. 62/905,952, filed on Sep. 25, 2019.

(51) Int. Cl. C12Q 1/6886

(52) U.S. Cl. CPC C12Q 1/6886; C12Q 2600/16; C12Q 2600/112; C12Q 2600/158

(57) ABSTRACT

An assay method is provided for determining the status of the health of a tissue within the body of a subject, wherein the tissue comprises an abundance of at least a first target protein, wherein assay comprises analysis of a liquid biopsy sample obtained from the subject, and wherein the liquid biopsy sample comprises at least a first cell free mRNA (cf-mRNA) that encodes the at least a first target protein. The assays and methods are useful in the diagnosis and treatment of liver health in a subject. In particular, methods and systems are provided for the diagnosis and treatment of fatty liver; non-alcoholic steatohepatitis (NASH); cirrhosis; liver disease; hepatitis; and liver cancer.

This application is a continuation of PCT/US2020/052208, filed Sep. 23, 2020; which claims the benefit of U.S. Provisional Application No. 62/905,952, filed Sep. 25, 2019. The contents of the above-identified applications are incorporated herein by reference in their entireties.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence Listing.txt with a creation date of Sep. 21, 2020, and a size of 767 bytes. The Sequence Listing filed via EFS-Web is part of the specification and is hereby incorporated in its entirety by reference herein.

### FIELD OF THE INVENTION

The present invention is directed towards liquid biopsy analysis of extracellular RNA as a prognostic, diagnostic or treatment tool based upon determination of health status within animals, such as humans.

### BACKGROUND OF THE INVENTION

According to the World Health Organisation, about 46% of global diseases and 59% of associated the mortality results from chronic diseases. This means that every year almost 35 million people in the world die of chronic diseases (Murray C J, Lopez A D. Evidence-based health policy—lessons from the Burden of Disease Study. Science 1996; 274: 740-743). As a proportion of chronic diseases, liver disease rates are steadily increasing over the years. The global burden of chronic liver disease and cirrhosis is substantial. Although vaccination, screening, and anti-viral treatment campaigns for infectious causes of liver disease have reduced the healthcare burden in some more developed nations, increases in injection drug use, alcohol misuse, obesity and metabolic syndrome threaten these efforts. Consequently, liver diseases are recognized as the second leading cause of mortality amongst all gastroenterological diseases in the US.

It is estimated that by 2015 around 600 million adults in the world were clinically obese (A. Afshin, M. H. Forouzanfar et al., New England Journal of Medicine, vol. 377, no. 1, pp. 13-27, 2017). As populations age and levels of obesity rise across the globe the prevalence of chronic conditions such as non-alcoholic fatty liver disease (NAFLD) has risen. NAFLD refers to a group of conditions in which there is fat accumulation in the liver without liver cell injury in people who do not have excessive alcohol use. Surprisingly the prevalence of NAFLD is been estimated to be as high as 25% in the general population and even higher (>70%) in patients with other metabolic risk factors like obesity and diabetes (Lazo, M et al. Prevalence of Non-alcoholic Fatty Liver Disease in the United States: The Third National Health and Nutrition Examination Survey, 1988-1994. Am. J. Epidemiol. 2013, 178, 38-45).

Chronic liver disease may often be relatively asymptomatic in its early stages but as many as 50% of patients with NAFLD will further progress and develop non-alcoholic steatohepatitis (NASH), a much more severe form of fatty liver disease. Patients who suffer from NASH can progress to the end-stages of chronic liver disease, including cirrhosis and hepatocellular carcinoma (HCC).

Patients with NASH can present with steatosis, hepatocellular injury, focal mixed cell-type inflammation, and fibrosis. The inflammatory response in the liver may cause cellular damage and progression of the disease over time. The acute phase response (APR) comprises a cascade of systemic responses upon tissue injury whereby the liver is the main target as a result of disease. Inflammatory processes are the main causes of the initiation of these defence mechanisms with a range of cytokines predominantly responsible as mediators for APR. Minimally invasive approaches have focused on assessing liver health by identifying the concentrations of APR cytokine proteins in samples of blood, a so-called ‘liquid biopsy’. However, attempts to correlate the concentrations of acute phase proteins in the blood with liver histology have not yielded clear outcomes (Koruk et al., Turk J Gastroenterol. 2003: 14 (I): 12-17).

An alternative approach to non-invasive evaluation of liver function involves the use of a LiMAx® test which assesses the enzymatic capacity of cytochrome P450 1A2 (Alizai et al. Gastroenterology Research and Practice, Volume 2019, Article ID 4307462, 7 pages). The LiMAx® test is based on ¹³C-hepatic methacetin metabolism by the cytochrome P450 1A2 system. After injection, methacetin is metabolized into acetaminophen and ¹³CO₂ which is pulmonary exhaled. A breath analysis is performed on the test subject with a bedside laser-based nondispersive isotope-selective infrared spectroscope device which allows a determination of liver function capacity to be made. The LiMAx® test is, of course, limited to determination of activity of a single cytochrome P450 so assessment of liver function is an extrapolation from a single enzyme activity. It also relies on the use of specialist equipment and reagents which need to be administered within a controlled environment. Finally, the LiMAx® test cannot be used for patients who are smokers due to interference with the assay.

The primary approach to diagnosing and treating NAFLD and NASH using non-invasive or minimally invasive approaches relies upon diagnostic imaging such as MRI and ultrasound (Madrazo, Gastroenterol Hepatol (N Y). 2017 June; 13(6): 378-380). However, the gold standard for diagnosing patients with NAFLD, especially NASH, is still considered to be liver biopsy. Clearly this invasive procedure is potentially painful and can be a complex procedure for many of the patient populations affected—e.g. elderly or bariatric patents. Hence, there is a need for improved minimally invasive methods for diagnosis of chronic liver diseases such as NAFLD and NASH.

Cell free nucleic acids are present in the bloodstream and include RNA, so-called ‘circulating RNA’, despite the typically very short half-life of RNA outside cells (El-Hefnawy et al., Clin Chem, 2004). RNA molecules of this nature therefore are indicated to be associated with lipids, such as vesicles and lipoproteins, to enable their survival. Circulating RNA includes mRNA, which can be enriched in microvesicles or exosomes released by cells.

WO-A-02/00935 (Ramanathan) provides a description of a method for estimating the levels of certain drug metabolizing enzymes in liver—so-called “drug clearance markers”—by correlating to levels of mRNAs found in the blood cells of an individual. In Ramanathan, mRNA is isolated from a blood sample, and reverse transcribed to form cDNA, which is then analysed on a DNA microarray in order to estimate the presence and amount of protein levels of drug clearance markers in the liver based on the corresponding levels of hepatic mRNA expression. There are problems in the methodology of Ramanathan because it relies upon two assumptions:
1) that there is a direct correlation between the levels of mRNA in the blood with corresponding levels of mRNA for a given enzyme or transporter in the liver of that individual; and
2) that the individual liver mRNA levels correspond in a linear fashion to the amount of protein of the same liver enzymes and transporter present in that individual.

Ramanathan's own experiments rely on correlations between tested levels of mRNAs of different enzymes in blood samples from a first group of individuals with previously reported prior art measures of corresponding liver enzymes from a second group of different individuals. Hence, no meaningful correlations can be determined from the Ramanathan studies for any given specific enzyme and transporter as the alleged correlation occurs for a set of different enzymes and transporters between samples taken from blood and liver of different individuals. Indeed, many factors affect the translation of mRNAs into a given protein and that for any one given gene expression product there may be multiple regulatory mechanisms that control its translation into a corresponding functional protein. In the case of human-derived hepatocytes, for a several thousand-fold increase in mRNA, there might be only a few-fold change in the actual level of protein (as reviewed by Einolf et al, Clin Pharmacol Ther. 2014 February; 95(2):179-88). Such effects may also be highly susceptible to environmental, genetic and lifestyle factors that can modulate the level and activities of drug clearance enzymes in vivo on an individual basis.

A further complication arises from a phenomenon described as “shedding” of mRNA by the cells of an organ or tissue often within exosomes into bodily fluids, such as into the bloodstream. The amount of shedding varies between individuals with “fast shedders” releasing a higher amount of RNA for the same amount of transcription of a particular gene in the originating organ or tissue when compared to that released by “slow shedders”. It can be appreciated, therefore, that quantification of circulating RNA alone without correction for the level of shedding within an individual will be only of limited use in accurately predicting the protein levels derived from expression of a particular gene in organ tissue. Hence, assertions in the art that circulating mRNA, of exosomal or other origin, may serve as a source of “liquid biopsy” for correlation with abundance of organ drug handling proteins are at best speculative and at worst highly premature in addressing the significant technical problems that exist.

International Patent Application No. PCT/US2019/24379 provides a liquid biopsy technology platform for transcriptomics analysis of a sample of bodily fluid taken from a human or animal subject (e.g. blood) to determine the concentration of one or more target mRNAs in the sample. The concentration of the target mRNA is adjusted with a shedding correction factor that compensates for an individual subject's level of mRNA shedding for the organ/tissue source of the target mRNA. The concentration of the one or more target mRNAs in the liquid biopsy sample may be correlated to protein abundance and, thus, functional activity in the source organ/tissue.

It would be desirable to provide improved methods, assays for the identification and treatment of diseases in patients using minimally invasive liquid biopsy technologies.

These and other uses, features and advantages of the invention should be apparent to those skilled in the art from the teachings provided herein,

### SUMMARY OF THE INVENTION

Accordingly, a first aspect of the invention provides an in vitro assay method for determining the status of the health of a tissue within the body of a subject, wherein the tissue comprises an abundance of at least a first target protein, wherein the assay comprises analysis of a liquid biopsy sample obtained from the subject, and wherein the liquid biopsy sample comprises at least a first cell free messenger RNA (cf-mRNA) that encodes the at least a first target protein, the method comprising:
1. isolating the at least a first cf-mRNA from the liquid biopsy sample;
2. ascertaining the abundance of the first target protein within the tissue within the subject, by identifying the concentration of the first cf-mRNA in the liquid biopsy sample, wherein the concentration of the first cf-mRNA in the liquid biopsy sample is normalized for the subject in order to correct for nucleic acid shedding in the subject, and the normalized concentration of the first cf-mRNA is correlated to the abundance of the first target protein in the tissue by a standard function; and
3. categorizing the health of the tissue within the body of the subject by analysing the abundance of the first target protein in the tissue.

In a second aspect, the invention provides a method for treating a diseased tissue within the body of a subject, wherein the diseased tissue comprises an abundance of at least a first target protein, wherein the method comprises analysis of a liquid biopsy sample obtained from the subject, and wherein the liquid biopsy sample comprises at least a first cell free messenger RNA (cf-mRNA) that encodes the at least a first target protein, the method comprising:
i isolating the at least a first cf-mRNA from the liquid biopsy sample;
ii ascertaining the abundance of the first target protein within the tissue within the subject, by identifying the concentration of the first cf-mRNA in the liquid biopsy sample, wherein the concentration of the first cf-mRNA in the liquid biopsy sample is normalized for the subject in order to correct for mRNA shedding in the subject, and the normalized concentration of the first cf-mRNA is correlated to the abundance of the first target protein in the tissue by a standard function;
iii categorizing the health of the tissue within the body of the subject by analysing the abundance of the first target protein in the tissue; and
iv treating the subject with medication if the tissue is identified as diseased.

A third aspect of the invention provides for a system for assessing the status of the health of a tissue within the body of a subject, the system comprising:
a receptacle for receiving a liquid biopsy sample obtained from the subject, wherein the liquid biopsy sample comprises at least a first cell free mRNA (cf-mRNA) that encodes a first target protein, and wherein the first target protein is a protein that is expressed in the tissue and the concentration of the first target protein in the tissue is indicative of the health status of the tissue;
a process module for processing the liquid biopsy sample to quantify the amount of first cf-mRNA present, wherein the process module comprises liquid handling apparatus for isolating and quantifying total cf-mRNA from the liquid biopsy sample; and
at least one analytics module for determining the concentration of the first cf-mRNA in the liquid biopsy sample, wherein the analytics module is in communication with the process module, the at least one analytics module comprising at least one controller wherein the controller performs a normalization function in order to correct for mRNA shedding in the subject, and the normalized concentration of the first cf-mRNA is further associated by the controller to the abundance of the first target protein in the tissue by a standard correlation function;
an interface module for providing an output to a user of the system, wherein the interface module provides a categorization of the health of the tissue within the body of the subject.

A fourth aspect of the invention provides a method of treating an individual subject in need thereof, wherein the individual is the intended recipient of a pharmaceutical treatment, the method comprising establishing a personalised virtual model of pharmaceutical compound exposure in the body of the individual subject prior to treatment, the process comprising the steps of:
isolating total cell free RNA (cfRNA_{TOTAL}) from a liquid biopsy obtained from the individual subject;
quantifying an amount of a first cell free RNA (cfRNA) present in the liquid biopsy, wherein the first cfRNA originates from a tissue in the body of the subject, and wherein the first cfRNA encodes a protein that is involved in pharmaceutical compound clearance or transport;
performing an adjustment function on the amount of the first cfRNA so as to correct for inherent levels of RNA shedding in the individual subject;
identifying the abundance of the protein within the organ/tissue of the subject by comparison of the corrected amount of the first cfRNA with abundance data for the corresponding amount of protein in the liver of the subject;
determining a pharmaceutical compound clearance or transport capacity for the individual subject based upon the abundance of the protein within the organ/tissue of the subject;
generating the personalised virtual model of pharmaceutical compound exposure based upon the pharmaceutical compound clearance or transport capacity of the individual subject; and
treating the individual with a dosage of pharmaceutical compound that is optimized to the individual based upon their personalised compound clearance capacity.

In embodiments of the invention the adjustment function comprises identifying the amount of the first cfRNA present by correcting against a RNA organ Shedding Correction Factor (SCF) that is determined for the individual subject by:
performing an analysis of the cfRNA_{TOTAL} in order to quantify an amount of mRNA present within the cfRNA_{TOTAL} that corresponds to each of two or more marker genes, wherein a marker gene is defined as a gene that is expressed principally and consistently in the organ/tissue; and
determining SCF as the mean concentration of mRNA of the each of two or more marker genes present within the cfRNA_{TOTAL}.

In one embodiment of the invention, the SCF is determined for the subject by isolating cfRNA_{TOTAL} from a liquid biopsy obtained from an individual subject, performing an analysis of the cfRNA_{TOTAL} in order to quantify an amount of two or more marker genes mRNAs present, designated as [cfRNA]_{Marker}, wherein a marker gene is defined as a gene that is expressed principally and consistently in the organ/tissue and at a relatively high level within the dynamic range of expression specific to the organ/tissue; and determining the SCF according to the formula A:

$$SCF = 1 {0}^{6} \cdot {\sum }_{i = 1}^{N} {[ cfRNA ]}_{{Marker}_{i}} / ( N \times {[ cfRNA ]}_{TOTAL} ) \qquad A$$

where N is equal to the number of marker genes quantified.

Suitably, at least three, suitably at least five, typically at least eight and optionally at least ten or more marker genes are selected in order to determine the SCF. Optionally, the organ is selected from one or more of the group consisting of: the liver; the kidney; the gut (e.g. G.I. tract); the brain/CNS; and the pancreas. In a specific embodiment the organ is the liver.

Where the organ/tissue is or comprises the liver, then at least one of the two or more marker genes may be selected from the group consisting of: A1BG (Alpha-1-B glycoprotein); AHSG (alpha-2-HS-glycoprotein); ALB (Albumin); APOA2 (Apolipoprotein A-II); C9 (Complement component 9); CFHR2 (Complement factor H-related 5); F2 (Coagulation factor II (thrombin)); F9 (Coagulation factor IX); HPX (Hemopexin); SPP2 (Secreted phosphoprotein 2); TF (Transferrin); MBL2 (mannose-binding lectin (protein C) 2); SERPINC1 (Serpin peptidase inhibitor, clade C (antithrombin), member 1); and FGB (Fibrinogen beta chain).

Where the organ/tissue is or comprises the gut the at least one of the two or more marker genes may be selected from the group consisting of: FABP6 (fatty acid binding protein 6); VIL1 (villin 1); LCT (lactase); DEFA6 (defensin alpha 6); DEFA5 (defensin alpha 5); CCL25 (C-C motif chemokine ligand 25); RBP2 (retinol binding protein 2); APOA4 (apolipoprotein A4); REG3A (regenerating family member 3 alpha); FABP6 (fatty acid binding protein 6); MEP1B (meprin A subunit beta); ALPI (alkaline phosphatase, intestinal); and CPO (carboxypeptidase O).

Where the organ/tissue is or comprises the brain/CNS at least one of the two or more marker genes may be selected from the group consisting of: OPALIN (oligodendrocytic myelin paranodal and inner loop protein); GFAP (glial fibrillary acidic protein); OMG (oligodendrocyte myelin glycoprotein); OLIG1/2 (oligodendrocyte transcription factor 1/2); GRIN1 (glutamate ionotropic receptor NMDA type subunit 1); NEUROD6 (neuronal differentiation 6); CREG2 (cellular repressor of E1A stimulated genes 2); NEUROD2 (neuronal differentiation 2); ZDHHC22 (zinc finger DHHC-type containing 22; KCNJ9 (potassium voltage-gated channel subfamily J member 9); GPM6A (glycoprotein M6A); PLP1 (proteolipid protein 1); and MBP (myelin basic protein).

Where the organ/tissue is or comprises the kidney at least one of the two or more marker genes may be selected from the group consisting of: UMOD (uromodulin); KCNJ1 (potassium voltage-gated channel subfamily J member 1); TMEM174 (transmembrane protein 174); NPHS2 (podocin); AQP2 (aquaporin 2); TMEM52B (transmembrane protein 52B); CTXN3 (Cortexin 3); TMEM27 (transmembrane protein 27); SOST (sclerostin); and CALB1 (Calbindin 1).

In particular embodiments of the invention the virtual model comprises a physiologically based pharmacokinetic (PBPK) model.

Typically, the first cfRNA encodes an organ protein. Suitably, the organ-derived cfRNA encodes a xenobiotic handling protein selected from the group consisting of: a xenobiotic clearance protein; a xenobiotic metabolising enzyme; and a xenobiotic transporting protein.

In embodiments of the invention, the xenobiotic is a pharmaceutical compound or drug. Optionally, the first cfRNA encodes an enzyme. In one embodiment of the invention, the enzyme comprises a cytochrome P450 monooxygenase (CYP) protein. The CYP protein may be selected from at least one of the group consisting of: Cytochrome P450 family 1 (CYP1); Cytochrome P450 family 2 (CYP2); Cytochrome P450 family 3 (CYP3); Cytochrome P450 family 4 (CYP4); Cytochrome P450 family 5 (CYP5); Cytochrome P450 family 7 (CYP7); Cytochrome P450 family 8 (CYP8); Cytochrome P450 family 11 (CYP11); Cytochrome P450 family 17 (CYP17); Cytochrome P450 family 19 (CYP19); Cytochrome P450 family 20 (CYP20); Cytochrome P450 family 21 (CYP21); Cytochrome P450 family 24 (CYP24); Cytochrome P450 family 26 (CYP26); Cytochrome P450 family 27 (CYP27); Cytochrome P450 family 39 (CYP39); Cytochrome P450 family 46 (CYP46); Cytochrome P450 family 51 (CYP51). Suitably, the CYP is selected from one of the group consisting of: CYP1A1; CYP1A2; CYP1B1; CYP2A6; CYP2A7; CYP2A13; CYP2B6; CYP2C8; CYP2C9; CYP2C18; CYP2C19; CYP2D6; CYP2E1; CYP3A4; CYP3A5; and CYP3A7. In a further embodiment, the enzyme comprises a transferase selected from one of the group consisting of: a methyltransferase; a sulfotransferase; an N-acetyltransferase; a glucuronosyltransferase including, but not limited to, one or more of the group consisting of UGT1A1, UGT1A3, UGT1A4, UGT1A6, UGT1A9, UGT2B4, UGT2B7, UGT2B15 and UGT2B17; a glutathione-S-transferase; and a choline acetyl transferase.

In another embodiment, the transport protein is an ATP-binding cassette (ABC) transporter or a solute carrier (SLC) transporter.

According to embodiments of the invention, the liquid biopsy comprises a sample of a bodily fluid selected from the group consisting of: blood; urine; saliva; semen; tears; lymphatic fluid; cerebrospinal fluid; bile; stool; pleural effusion; ascitic fluid; and a mucus secretion. In embodiments where the liquid biopsy comprises blood or a component thereof, it may comprise whole blood, serum and/or plasma.

In further embodiments of the invention the methods described provide for quantifying the amount of at least a second cell free RNA (cfRNA), or a third, fourth, fifth, sixth, seventh or more cfRNAs present in the liquid biopsy. In a specific embodiment, a plurality of cfRNAs are quantified each one of the plurality of cfRNAs corresponding to a different organ/tissue protein as defined herein.

According to embodiments of the invention determination of the clearance capacity for the individual subject based upon the abundance of the protein within the organ/tissue of the subject is achieved by use of an abundance curve or pre-determined function.

It will be appreciated that the features of the invention may be subjected to further combinations not explicitly recited above.

### DRAWINGS

The invention is further illustrated by reference to the accompanying drawings in which:

FIG. 1 shows an illustration of mRNA shedding from an organ, in this instance the liver is shown as the tissue of origin.

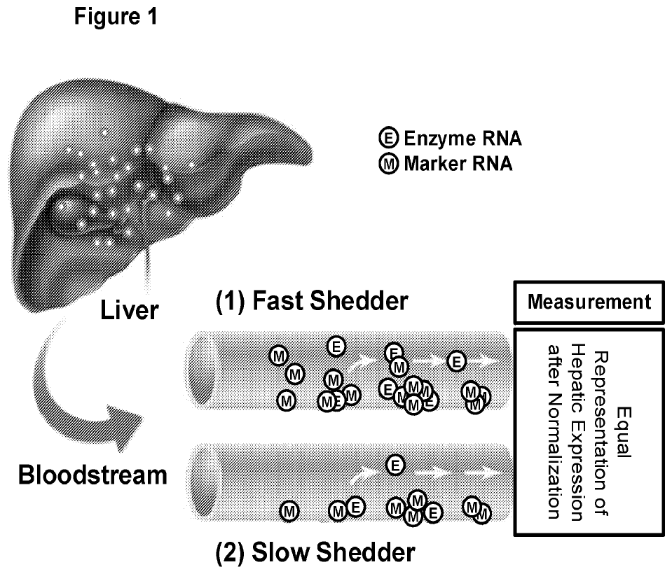

FIG. 2 shows a schematic of an embodiment of a system of the invention that creates an in silico virtual twin model of an individual subject based upon data obtained from a liquid biopsy from the subject in combination with computer based models.

FIG. 3 shows a schematic of an embodiment of a system of the invention that creates an in silico virtual twin model of an individual subject based upon data obtained from a liquid biopsy from the subject in combination with computer based simulation models. The data is then used to establish a personalised dosage regimen for a therapeutic treatment to be administered to the subject.

FIG. 4 shows a bar graph of plasma RNA levels which have been adjusted to accommodate shedding and corresponding protein abundance within matched samples from a group of individuals.

FIG. 5 shows graphs that indicate the correlation between plasma RNA levels which have been adjusted to accommodate shedding and the corresponding levels of protein expression in the tissue of origin. In this instance the plasma RNA encodes a range of CYPs.

### DETAILED DESCRIPTION OF THE INVENTION

Unless otherwise indicated, the practice of the present invention employs techniques of chemistry, computer science, statistics, molecular biology, microbiology, recombinant DNA technology, and chemical methods, which are within the comprehension of a person of ordinary skill in the art. Such techniques are also explained in the literature, for example, T. Cormen, C. Leiserson, R. Rivest, 2009, Introduction to Algorithms, 3rd Edition, The MIT Press, Cambridge, Mass.; L. Eriksson, E. Johansson, N. Kettaneh-Wold, J. Trygg, C. Wikstom, S. Wold, Multi- and Megavariate Data Analysis, Part 1, 2nd Edition, 2006, UMetrics, UMetrics AB, Sweden; M. R. Green, J. Sambrook, 2012, Molecular Cloning: A Laboratory Manual, Fourth Edition, Books 1-3, Cold Spring Harbor Laboratory Press, Cold Spring Harbor, N.Y.; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

An embodiment of the present invention provides a liquid biopsy technology platform for transcriptomics analysis of a sample of bodily fluid taken from a human or animal subject (e.g. blood) to determine the concentration of one or more target mRNAs in the sample. The concentration of each target mRNA is adjusted with a shedding correction factor that compensates for an individual subject's level of mRNA shedding for the organ/tissue source of the target mRNA. The concentration of the one or more target mRNAs in the liquid biopsy sample may be correlated to protein abundance and, thus, functional activity in the source organ/tissue.

Present inventors have identified a correlation between an organ or tissues' health status and the abundance of proteins that are involved in absorption (rate and extent of bioavailability), distribution, metabolism and excretion (ADME) in that organ or tissue. In a specific embodiment, the organ is the liver and may include liver disease (cirrhosis, NASH, hepatitis, fatty liver, NAFLD etc), but also inflammatory diseases (rheumatoid arthritis, HIV, viral hepatitis etc) and even hypertension. It is known that inflammation produces cytokines such as IL-6, which is an inhibitor of CYP3A4 activity. Therefore, the inventors have noted that changes in the 3A4 phenotype may be non-specific and be a result of many disease processes, similar to inflammatory markers like C-reactive protein (CRP). Further, CYP4A1 abundance and activity may be linked to essential hypertension. Of course, the expression and activity of CYPs may be affected by many factors, including endogenous and exogenous factors many of which have an effect on health status. Hence, providing a non-invasive assay method and system that can accurately assess the relative abundance of a plurality of ADME proteins within the body of a subject from a single liquid biopsy allows for surprisingly informative and accurate evaluation of organ and tissue health status.

The liquid biopsy platform represents a route to determining the status of the health of organs/tissues which express ADME proteins, such as CYPs, at significant levels—e.g. in the liver, gut and lung. In particular embodiments of the invention the determination of the function and health of the liver is the focus. Additionally, a non-CYP ADME protein, such as UDP glucuronosyltransferase 1 (UGT1A1 in humans), is also a useful target mRNA that can be indicative of biliary disease.

Prior to setting forth the invention in detail, definitions are provided that will assist in the understanding of the invention. All references cited herein are incorporated by reference in their entirety. Unless otherwise defined, all technical and scientific terms used herein have the same meaning as commonly understood by one of ordinary skill in the art to which this invention belongs.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may include DNA and RNA, subtypes of these such as genomic DNA, mRNA, miRNA, tRNA and rRNA. In embodiments of the invention mRNA is isolated from a liquid biopsy sample. Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers or probes for use in manipulation or detection of DNA such as via polymerase chain reaction (PCR).

The term “amino acid” in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties that are characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

A “polypeptide” is a polymer of amino acid residues joined by peptide bonds, whether produced naturally or in vitro by synthetic means. Polypeptides of less than around 12 amino acid residues in length are typically referred to as “peptides” and those between about 12 and about 30 amino acid residues in length may be referred to as “oligopeptides”. The term “polypeptide” as used herein denotes the product of a naturally occurring polypeptide, precursor form or proprotein. Polypeptides can also undergo maturation or post-translational modification processes that may include, but are not limited to: glycosylation, proteolytic cleavage, lipidization, signal peptide cleavage, propeptide cleavage, phosphorylation, and such like. The term “protein” is used herein to refer to a macromolecule comprising one or more polypeptide chains.

As used herein the term “biomarkers” may comprise cells, cellular components, peptides, polypeptides proteins, ncRNA, genomic DNA, metabolites, cytokines, antigens, and polysaccharides; as well as physiological parameters such as cell count, temperature, O₂ level, CO₂ level, or pH. Biomarkers may also comprise mRNA coding for polypeptides that are not involved in xenobiotic clearance. Suitably, the biomarkers comprise a combination of features.

The term “levels” is used herein to define terms of quantity or abundance of a specified factor and may be defined in molar or absolute amounts (i.e. micrograms or milligrams etc.), concentration (e.g. mg ml⁻¹ or mol g⁻¹ etc.), and/or in terms of a specific activity (e.g. units of activity in a standard assay). The selected “level” will be appreciated as appropriate to a given factor, for example, where it is appropriate to define the amount of a given enzymatic factor by its specific activity, it may be that this measure is selected rather than the actual amount (in mg/ml) of that factor that may be present. The term “normal level”, when in the context of levels of gene or polypeptide expression, is used herein to denote the level of gene expression or enzymic activity in healthy non-diseased comparator individuals, organs, tissue or samples. Normal levels of expression or activity represent the baseline or control range or level of expression of a gene. Aberrant levels in cells, either at levels that exceed the normal range within controls or that are too low, are considered not to be normal and can be indicative of disease in the samples from which the cells have been obtained, e.g. inflammation, infection or cancer. The term “high” in relation to levels may refer to strong, consistent and/or readily detectable expression and may not be considered as necessarily aberrant.

The term “allelic variant” is used herein to denote any two or more alternative forms of a gene occupying the same chromosomal locus and controlling the same inherited characteristic. Allelic variation arises naturally though mutation and may result in phenotypic polymorphism within populations. Gene mutations typically result in an altered nucleic acid sequence and in some cases an altered polypeptide sequence also. As used herein, the term “allelic variant” is additionally used to refer to the protein or polypeptide encoded by the allelic variant of a gene.

The term “isolated”, when applied to a polynucleotide sequence, denotes that the sequence has been removed from its natural organism of origin and is, thus, free of extraneous or unwanted coding or regulatory sequences. The isolated sequence is suitable for use in recombinant DNA processes and within genetically engineered protein synthesis systems. Such isolated sequences include cDNAs and genomic clones. The isolated sequences may be limited to a protein encoding sequence only (e.g. an mRNA) or can also include 5′ and 3′ regulatory sequences such as promoters, transcriptional terminators and UTRs.

The term “isolated”, when applied to a polypeptide is a polypeptide that has been removed from its natural organism of origin. It is preferred that the isolated polypeptide is substantially free of other polypeptides native to the proteome of the originating organism. It is most preferred that the isolated polypeptide be in a form that is at least 95% pure, more preferably greater than 99% pure. In the present context, the term “isolated” is intended to include the same polypeptide in alternative physical forms whether it is in the native form, denatured form, dimeric/multimeric, glycosylated, crystallised, or in derivatized forms.

As used herein, the term “organ” is synonymous with an “organ system” and refers to a combination of tissues and/or cell types that may be compartmentalised within the body of a subject to provide a biological function, such as a physiological, anatomical, homeostatic or endocrine function. Suitably, organs or organ systems may mean a vascularized internal organ, such as a liver, kidney, brain, gut or pancreas; or may comprise fluid organ systems such as the blood and circulatory system. Typically organs comprise at least two tissue types, and/or a plurality of cell types that exhibit a phenotype characteristic of the organ. In contrast “tissue” refers to an aggregation or population of cells of the same or a similar type and/or lineage that may cooperate with other tissues to form an organ system.

The term “sample” is used to describe isolated materials of biological origin that can be used for a diagnostic, analytical or prognostic purpose. Biological materials may be analysed in tissue microarrays, or via other assay methods, and can include tissues from specific organs such as liver, kidney, brain, heart, epithelium, lung, and bone, as well as other tissues; as well as fluid materials such as whole blood, plasma, serum, lymph, urine, stool, cerebrospinal fluid and saliva etc. Such materials may also include in vivo and in vitro cellular materials such as healthy or diseased cells, tissues and cell lines—e.g. cancer cell lines, which may be manipulated for in vitro purposes—e.g. immortalised cell lines or induced pluripotent stem cells. The macromolecules analysed in these materials typically include polypeptides such as proteins as well as polynucleotides such as RNA (including mRNA), and DNA.

The term “blood sample” may refer to any or all of whole blood, plasma, serum, erythrocyte and/or leucocyte fractions, and any other blood derivative. Blood samples may be comprised within a liquid biopsy obtained from an individual or plurality of individuals.

The term “microsome” refers to vesicles made by re-forming of the endoplasmic reticulum (ER) during the break-up of cells in vitro, which can be concentrated and isolated from other cell debris. Cytochrome P450 monooxygenase enzymes (CYPs) are present in ER and so microsomal preparations containing CYPs can be obtained from tissue samples such as organ tissue (e.g. liver), where CYPs are highly abundant. CYPs are further discussed below.

The term “microvesicle” or “exosome” relates to extracellular vesicles that may be produced or shed by cells for example by exocytosis, budding or blebbing of the plasma membrane. Cell death by apoptosis may also lead to microvesicle production. Microvesicles are found in interstitial space and in many body fluids, and may contain mRNA, miRNA and/or proteins. It is thought that methods of intercellular communication may rely on microvesicle transport. Exosomes are a type of microvesicle that range in size from nanometer scale through to micrometer size. Exosomes are derived from parental cells comprised within organs or tissues so they are able to reflect both the physiological and pathophysiological state of those parental cells.

“Cell free nucleic acid” may be DNA, RNA, or any combination thereof. The nucleic acid may be cell free DNA (cfDNA), cell free RNA (cfRNA), or any combination thereof. The samples from which the cell free nucleic acids may be isolated include any bodily fluid capable of providing a liquid biopsy. Where the liquid biopsy comprises blood, the cell free nucleic acids may be located within plasma or serum.

As used herein, the phrases “drug metabolizing enzymes” or “ADME proteins” will include cytochrome P450 monooxygenase enzymes (CYPs) as well as membrane transport proteins, and transferases. The CYP protein may be selected from at least one of the group consisting of: Cytochrome P450 family 1 (CYP1); Cytochrome P450 family 2 (CYP2); Cytochrome P450 family 3 (CYP3); Cytochrome P450 family 4 (CYP4); Cytochrome P450 family 5 (CYP5); Cytochrome P450 family 7 (CYP7); Cytochrome P450 family 8 (CYP8); Cytochrome P450 family 11 (CYP11); Cytochrome P450 family 17 (CYP17); Cytochrome P450 family 19 (CYP19); Cytochrome P450 family 20 (CYP20); Cytochrome P450 family 21 (CYP21); Cytochrome P450 family 24 (CYP24); Cytochrome P450 family 26 (CYP26); Cytochrome P450 family 27 (CYP27); Cytochrome P450 family 39 (CYP39); Cytochrome P450 family 46 (CYP46); Cytochrome P450 family 51 (CYP51). In embodiments of the invention the CYP enzymes are selected from human CYP families 1, 2 and 3, which are the CYP families most typically linked to xenobiotic (e.g. drug) metabolism and clearance (i.e. ADME). Suitably the CYPs may comprise any, some or all of the CYPs selected from the group consisting of: CYP1A1; CYP1A2; CYP1B1; CYP2A6; CYP2A7, CYP2A13; CYP2B6; CYP2C8; CYP2C9; CYP2C18; CYP2C19; CYP2D6; CYP2E1; CYP3A4; CYP3A5 and CYP3A7. CYPs are haemoproteins, that is, of the superfamily of proteins containing haem (or heme) as a cofactor. These proteins are involved in the metabolism of xenobiotics, in general by oxidation reactions involving NADPH and oxygen. Different drugs often have different CYP proteins involved in their metabolism, a selection of exemplary compounds that are substrates for corresponding metabolizing CYPs are listed below—it will be appreciated that this list is non-exhaustive:—

| | |
|---|---|
| CYP1A2 | Caffeine; Tacrine; Theophylline; Melatonin; |
| | Clozapine; Lidocaine |
| CYP2A6 | Bilirubin; Cortinine; Coumarin |
| CYP2B6 | Benzphetamine; Buproprion; Efavirenz |
| | Methamphetamine; Temazepam; |
| CYP2C8 | Amodiaquine; Paclitaxel; Ibuprofen |
| CYP2C9 | Diclofenac; Irbesartan; Valsartan; Ibuprofen; |
| | Tamoxifen; Tolbutamide |
| CYP2C19 | Hexobarbital; Imipramine; Melatonin; |
| | Omeprazole; Diazepam |
| CYP2D6 | Codeine; Dihydrocodeine; Amphetamine; |
| | Loratidine; Oxycodone; Paroxetine; |
| | Risperidone; Tamoxifen |
| CYP2E1 | Aniline; Chlorzoxasone; Halothane; |
| | Isoflurane; para-Nitrophenol; Vinyl chloride |
| CYP3A4/5 | Alfentanil, Alprazolam; Atorvastin; Cortisol; |
| | Cholesterol; Dasatinib; Dexamethasone; |
| | Diazepam; Midazolam; Prednisolone; |
| | Quinine; Sildenafil; Testosterone; |
| | Triazolam; Vincristine |

(Zanger & Schwab (2013) Pharmacology & Therapeutics 138 (2013) 103-141; Watari et al. (2019) Biol. Pharm. Bull. 42, 348-353);

The CYP3A subclass catalyses an extensive number of oxidation reactions of clinically important drugs as shown above. It is currently believed that greater than 60% of clinically used drugs are metabolized by the CYP3A4 enzyme, including several major drug classes. Hence, accurately determining the abundance of even CYP3A4 alone in the liver of an individual subject based upon a liquid biopsy would facilitate the development of virtual PBPK models that would be able to predict that individual's capacity for clearance of dozens of approved drugs currently on the market.

Other, non-CYP, ADME proteins that are involved in metabolism of xenobiotic molecules include transferases: enzymes that catalyse the transfer of a functional group from a donor molecule to a specified substrate molecule (an acceptor) which is typically a drug or other xenobiotic compound. Transferase enzymes involved in drug metabolism are typically those that catalyse conjugation of moieties such as glutathione, methyl groups, acetyl groups, sulfate, and amino acids to a substrate molecule which may include a drug or a metabolite of a drug. Exemplary drug metabolizing transferases may include methyltransferases; sulfotransferases; N-acetyltransferases; glucuronosyltransferases (UDP-glucuronosyltransferases or UGTs) including, but not limited to, one or more of the group consisting of UGT1A1, UGT1A3, UGT1A4, UGT1A6, UGT1A9, UGT2B4, UGT2B7, UGT2B15 and UGT2B17; glutathione-S-transferases; and choline acetyl transferases.

In addition to the above, membrane bound and non-membrane bound transport proteins also may influence the levels of xenobiotic compound uptake and, hence, the levels of metabolism and clearance of a given compound within the body of an individual. Transport proteins may include one or more of the group selected from: transmembrane pumps, transporter proteins, escort proteins, acid transport proteins, cation transport proteins, vesicular transport proteins and anion transport proteins. Exemplary transporter proteins include ATP-binding cassette (ABC) transporters including, but not limited to, one or more of the group selected from: ABCB1/MDR1, ABCB11/BSEP, ABCC2/MRP2, ABCG2/BCRP. Alternatively, solute carrier (SLC) transporters may include one or more of the group consisting of: SLCO1B1/OATP1B1, SLCO1B3/OATP1B3, SLCO1A2/OATP1A2, SLCO2B1/OATP2B1, SLC22A1/OCT1, SLC22A7/OAT2, and SLC47A1/MATE1.

As used herein, the phrases “organ marker genes” or “marker genes” refer to genes expressed principally in organs associated with drug/xenobiotic clearance, suitably the liver, consistently and at relatively high levels. By “relatively high levels” it is meant that the expression profile of a given marker gene is expressed, usually constitutively, at readily detectable and quantifiable levels. In the present invention, the amount of these marker genes as measured in circulating RNA may be used as an indicator for the degree of shedding taking place in a certain individual. Thus these data on marker gene level may be used as a ‘benchmark’ to determine an average baseline of shedding in an individual, showing a basal level of organ gene expression. Such data may be used to reduce variation in correlation of other mRNA sample levels to expression levels of genes in the organ.

Suitably, organ marker genes indicative of the liver may be selected from any, some or all of: A1BG (Alpha-1-B glycoprotein), AHSG (alpha-2-HS-glycoprotein), ALB (Albumin), APOA2 (Apolipoprotein A-II), C9 (Complement component 9), CFHR2 (Complement factor H-related 5), F2 (Coagulation factor II (thrombin)), F9 (Coagulation factor IX), HPX (Hemopexin), SPP2 (Secreted phosphoprotein 2), TF (Transferrin), MBL2 (mannose-binding lectin (protein C) 2), SERPINC1 (Serpin peptidase inhibitor, clade C (antithrombin), member 1) and FGB (Fibrinogen beta chain).

Organ marker genes indicative of the gut include: FABP6 (fatty acid binding protein 6); VIL1 (villin 1); LCT (lactase); DEFA6 (defensin alpha 6); DEFA5 (defensin alpha 5); CCL25 (C-C motif chemokine ligand 25); RBP2 (retinol binding protein 2); APOA4 (apolipoprotein A4); REG3A (regenerating family member 3 alpha); FABP6 (fatty acid binding protein 6); MEP1B (meprin A subunit beta); ALPI (alkaline phosphatase, intestinal); and CPO (carboxypeptidase O); those indicative of CNS include: OPALIN (oligodendrocytic myelin paranodal and inner loop protein); GFAP (glial fibrillary acidic protein); OMG (oligodendrocyte myelin glycoprotein); OLIG1/2 (oligodendrocyte transcription factor 1/2); GRIN1 (glutamate ionotropic receptor NMDA type subunit 1); NEUROD6 (neuronal differentiation 6); CREG2 (cellular repressor of E1A stimulated genes 2); NEUROD2 (neuronal differentiation 2); ZDHHC22 (zinc finger DHHC-type containing 22; KCNJ9 (potassium voltage-gated channel subfamily J member 9); GPM6A (glycoprotein M6A); PLP1 (proteolipid protein 1); and MBP (myelin basic protein)]; and those specific to kidney include: UMOD (uromodulin); KCNJ1 (potassium voltage-gated channel subfamily J member 1); TMEM174 (transmembrane protein 174); NPHS2 (podocin); AQP2 (aquaporin 2); TMEM52B (transmembrane protein 52B); CTXN3 (Cortexin 3); TMEM27 (transmembrane protein 27); SOST (sclerostin); and CALB1 (Calbindin 1).

It will be appreciated that the aforementioned lists are not exhaustive and a plurality of alternative organ or tissue specific marker genes may be selected from, for example, the organ-specific or tissue-specific proteomes respectively. Often organ specific marker genes will comprise constitutively expressed genes that show relatively constant or consistent levels of expression with low or predictable variance over time in tissues having normal pathology—e.g. housekeeping genes. Organ or tissue phenotype marker genes may be comprised within a ‘panel’ that comprises a plurality of such genes. Typically, a panel of organ/tissue marker genes would include not less than six, suitably not less than eight, typically around ten and optionally not less than twelve genes expressed principally in the specified organ or tissue, consistently and at relatively high levels. Such marker genes may be derived from healthy tissues or organs, or they may be derived from diseased tissues/organs. In an embodiment of the invention, the tissue comprises neoplastic tissue, which may be benign or malignant.

As used herein the term “shedding” is used to describe the process of mRNA release by cells from organs or tissues, such as liver hepatocytes, into a bodily fluid, in microvesicles, exosomes, or otherwise as cell free mRNA. mRNA shedding can vary in magnitude between subjects or within the same subject depending on, for example, disease state, and affects the correlation between the levels of a particular RNA detected in the blood, plasma or other sample, and the levels of the same mRNA in the cells and tissue of the organ, such as the liver. The term “RNA shedding” is used as a synonym.

A “shedding coefficient”, organ “shedding correction factor” or “SCF” refers to a scaling factor for an individual which relates to the amount of shedding by their hepatocytes. A “fast shedder” will shed more RNA for the same amount of gene expression than will a “slow shedder”, and thus the SCF for such individuals will differ. It is contemplated that the SCF can be calculated from the quantified levels of the cell free RNA (cfRNA) of one or a plurality of organ/tissue marker genes, for example from 1, 2, 3, 4, 5, 6, 7, 8, 9, 10, 11, 12, 14 or more genes. The phenomenon of shedding correction is identified and exemplified in co-pending International Patent application No. PCT/US2019/24379 (the contents of which are incorporated herein by reference).

If a subset of N organ or tissue specific markers are used the SCF can be calculated as follows.

$$SCF = 1 {0}^{6} \cdot {\sum }_{i = 1}^{N} {[ cfRNA ]}_{{Marker}_{i}} / ( N \times {[ cfRNA ]}_{TOTAL} )$$

The SCF is expressed per million transcripts present in the cfRNA_{TOTAL} library created from the liquid biopsy.

Identification of the SCF for a given individual subject from which a liquid biopsy has been obtained is, therefore, a feature of embodiments of the present invention. The SCF allows for the further analysis of the exosomal mRNA in the biopsy in order to identify the abundance of a protein encoded by the mRNA in the organ/tissue of origin for the subject. Without the ability to correct for RNA shedding the derivation of transcriptomic information from any liquid biopsy is made substantially more difficult and potentially even meaningless.

Pharmacokinetics (PK) is the study of what happens to a drug when it is administered to and passes through the various organ and tissue compartments within the body of a subject. Drug absorption, distribution, and elimination are subject to multiple interactions dependent in part upon the biological action of each organ on a drug, partitioning of the drug to these organs and tissue volumes (compartments) and blood flows. The ADME and toxicity profiles of any given pharmaceutical or other xenobiotic compound are key deterministic measures of subsequent pharmacodynamics (action of the drug on body) necessary to achieve efficacy without major safety issues prior to an authorisation for use in medicine. Hence, the ADME capacity of organs and tissues can serve as an indication of health status and normal functioning. This is particularly evident in the liver which is a particularly important location for xenobiotic clearance and metabolism. Assessment of health status and normal functioning can inform treatment choices and methods for a wide variety of diseases and pathological conditions.

In an embodiment, the present invention is based in part upon an assay that determines the level of one or more mRNAs coding for xenobiotic metabolizing enzymes or clearance proteins originating from an organ/tissue in a liquid biopsy, such as a biological blood sample, via a quantitative analysis of the sample. This analysis establishes a correlation from these determined levels to the levels—or abundance/concentration—of xenobiotic metabolizing and/or transporting enzymes (e.g. ADME proteins) in the organ, and membrane transporters and transferase enzymes in other tissues of an individual. The abundance relationship may be supplemented by reference to standardized curves or log tables generated by comparison of matched samples comprising a liquid biopsy and a tissue biopsy from one or more reference individuals. Generally it is preferred that the matched samples are obtained from the same individual. In this way, the concentration of mRNA for a clearance or metabolizing protein present in the liquid biopsy is capable of direct relation to the corresponding abundance/concentration of the protein in the organ/tissue of origin. This in turn allows for the xenobiotic clearance capacity of the organ/tissue of origin to be estimated with a high degree of accuracy. Information regarding the xenobiotic ADME capacity of an organ or tissue for a given ADME protein (such as a CYP, transferase or transporter) represents one of the building blocks of bottom up PBPK models.

Variability of drug response between individuals is an important consideration in clinical medicine. One major determinant of drug response variability is hepatic CYP-mediated drug metabolism due to polymorphism and allelic variation, and difference in expression levels across populations. Sex differences are also known to affect drug metabolism with females more than 50 to 75% more likely to observe adverse effects. Other variations may be due to polymorphism or difference in expression levels of other key proteins involved in drug clearance including membrane transporter proteins and transferase enzymes. Hence, the analytical data generated by the inventive methods and apparatus provides a key advantage that enables the construction of significantly improved personalised as well as population based pharmacokinetic computer models. These models and simulations may be used in the design of improved clinical trials, incorporated into the decision of better dosage regimens, or used to predict and inform personalised medicine choices.

The individuals tested or treated according to various embodiments of the invention may be healthy or diseased, and human or animal patients. In veterinary contexts, the drug clearance models may require suitable adaptation, although the underlying principles of the invention are consistent. The term “animal” may include fish, birds, amphibians and reptiles as well as mammals such as cats; dogs; mice; guinea pigs; rabbits; primates; horses; as well as livestock including cattle; pigs; sheep; and goats.

For individuals or populations suffering from disease or abnormal pathology the current development of PBPK models is difficult due to a lack of available data to better characterize the altered physiological state. The lack of in vivo data for generating models for individuals and populations suffering from cancer is particularly evident. Prediction of drug clearance during infection and inflammation is a further important consideration for disease state modelling, given that this altered state leads to downregulation of metabolizing enzymes such as CYPs in the liver and gut due to elevated levels of pro-inflammatory cytokines. Since the design of personalised dosage regimens for drugs in individual patients is assigned based on the extent of drug clearance for that individual to avoid overexposure, the effect of disease such as renal or hepatic impairment, which can significantly reduce clearance, is crucial. Hence, in embodiments of the invention a process is provided for establishing a virtual PBPK model of metabolic xenobiotic clearance in an individual subject or a population of individual subjects, wherein the subject(s) are suffering from disease or altered physiological state associated with an abnormal pathology.

In an embodiment of the invention, the levels of mRNAs—suitably cell free mRNAs—that encode drug metabolizing and transporting ADME proteins and those involved in homeostasis (metabolism and disposition of endogenous compounds), including CYPs, transport proteins and transferases, are measured in a liquid biopsy, suitably a blood sample. The concentration or amount of each mRNA in the blood sample thereby correlates to an amount/concentration/abundance of a drug clearance protein, for example, an enzyme or transporter, in the organ or tissue of the individual from which the mRNA originated. The prediction of amount/concentration/abundance of an ADME protein based upon the amount or concentration of the mRNA present in the liquid biopsy can be made by consultation with a calibration curve or log table, for instance.

Liquid biopsy enables organ/tissue health to be assessed, particularly liver health, by:
1. Monitoring relative levels of abundance of target proteins (e.g. CYPs or UGT1A1) in the source tissue;
2. Determining whether ratios of relative abundance of target proteins are aberrant or have changed;
3. Predicting levels of activity of target proteins in the source tissue;
4. Identifying whether additional disease markers, such as inflammatory or cancer markers, are also expressed
5. Characterising any polymorphisms present in target proteins that may result in variation of activity or function.

The ability to screen for multiple ADME mRNA targets from a single liquid biopsy sample, thereby allowing for many more correlations to be made. There is also less possibility that the results will be impacted by the presence of compounds that might inhibit the activity of a single target protein such as CYP inhibitory concomitant medications, smoking, inflammatory inhibitors (e.g. acute phase reactants). The ongoing and continual monitoring of subjects is far easier if it requires simple liquid biopsy (e.g. sequential blood draw) rather than administration of metabolisable substances and monitoring for degradation such as in the LiMAx® test. This allows for the methods and assays of the present invention to operate on at least an out-patient or even a remote basis with minimal requirement for medical professional supervision of the subject.

In embodiments of the invention the ADME protein may be predominantly, or even exclusively, expressed within a single tissue or organ or origin. This enables a high level of confidence that the adjusted level of circulating mRNA determined according to the methods described correlate well to the abundance of the encoded protein in the tissue of origin. An example of a protein having relatively restricted expression is CYP1A2, which is predominantly expressed in the liver. In instances where a protein can be expressed by more than one tissue or organ of origin the calculated concentration level of that protein can be weighted accordingly in relation to the known levels of protein abundance in the tissues of origin. For example, CYP3A may be expressed in both the gut and the liver although the net intestinal abundance of CYP3A in the gut is only around 1% of that in the liver (Yang et al. Clinical pharmacology & therapeutics (2004); 76(4); and Yang et al. Current Drug Metabolism (2007) 8, 676-684).

In specific embodiments, the methods and assay systems of the invention can be used with both human and animal subjects including mammals, birds and fish. In such an embodiment the liquid biopsy may be used for veterinary purposes as well a measure of environmental contamination. For instance, monitoring of pollutant effects on health of fauna in vulnerable ecosystems can be achieved by taking liquid biopsy samples from captured animals and analysing them for multiple ADME mRNA targets according to the methods of embodiments of the invention.

According to a specific embodiment of the present invention the relative levels of ADME proteins in the tissue of origin may be assessed to identify particular correlations, ratios or other prognostic or diagnostic associations. In one embodiment, measurement of the ratio of concentration of at least a first CYP relative to at least a second CYP within the liver of a subject may be indicative of liver health and function. More specifically, this measurement may provide prognostic or diagnostic information of whether the subject individual exhibits aberrant pathology, such as any one of the multiple chronic liver diseases described previously. In further embodiments, measurement of the concentrations of multiple CYPs may provide for a prognostic or diagnostic ‘signature’ that emerges that provides added predictive value. Such measurements may feed in to accepted prognostic scores such as Child-Pugh criteria for liver diseases such as cirrhosis.

The transcriptomics profile can be used to build a virtual system to provide an in silico model for an individual subject or if combined with a plurality other individuals to provide virtual population, or sub-population. Such models can be tested to predict the individual or a population's capacity for clearance of one or more xenobiotic compounds. The system can be further refined by the addition of information derived from biomarkers found within the same or a different sample, and/or with other physiological and/or epidemiological information, which may be gathered by questionnaire, interview, health professional analysis, measurement with medical diagnostic equipment, or similar. The virtual individual or population can also be tested for undesirable interactions that might occur between combinations of xenobiotics. For example, in the field of pharmacology such interactions are referred to as drug-drug interactions (DDIs). Conventionally model based DDIs are studied by either a Mechanistic Dynamic interaction Model (MDM) based on in vitro data plugged into an appropriate PBPK model or a Mechanistic Static interaction Model based on in vivo data (IMSM). A problem with IMSM models is that they can be constrained by availability of reliable in vivo data. For instance, it is known that genetic polymorphism can have a significant effect upon cytochrome metabolism and, thus, upon any DDIs that may occur in an individual (Tod et al. AAPS J., 2013 October; 15(4): 1242-1252). Most IMSM models require multiple interactions of compounds to be undertaken in many individuals. By enabling the generation of additional in vivo data from a liquid biopsy, it is an advantage of the present invention that it allows for the development of better IMSMs as well as a hybrid approach in which an MDM can be further informed by real world in vivo data that feeds into and allows for the generation of highly bespoke PBPK models.

Isolation of exosomal or microvesicular components from a liquid biopsy may be performed using techniques such as spin column chromatography, immunoaffinity, membrane affinity, precipitation and/or ultracentrifugation with a density gradient. Optimisation or choice of techniques will depend upon factors such as sample volume versus the type/origin of liquid biopsy being handled. In an example of the invention described in more detail below, RNA comprised within exosomal or microvesicular components of a blood plasma liquid biopsy are isolated using a membrane affinity column utilising selective binding to a silica-based membrane. Precipitation methods may also be used to improve exosomal yield.

Biomarker levels within a liquid biopsy sample may be determined by a range of techniques including macromolecule microarray analysis, mass spectrometry (MS) proteomic profiling, quantitative RT-PCR, ELISA or other antibody-based assays, and chromatographic or spectrophotometric techniques.

RNA transcripts that are isolated from the liquid biopsy sample may be detected by a range of methods, including but not limited to polymerase chain reaction (PCR), reverse transcription polymerase chain reaction (RT-PCR), quantitative real time polymerase chain reaction (Q-PCR), gel electrophoresis, capillary electrophoresis, mass spectrometry, fluorescence detection, ultraviolet spectrometry, DNA hybridization, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof. RNA may be reverse-transcribed by any suitable means to produce cDNA before analysis in any combination with the above. RNA levels can be determined by use of nucleic acid hybridisation arrays, real-time PCR, or next generation sequencing techniques.

Bioanalysis of RNA samples may also occur using RNA sequencing such as by use of a single-end sequencing-by-synthesis reaction e.g. Ampliseq (Thermo Fisher, USA), HiSeq 2500 or NextSeq 550Dx Systems (Illumina, USA).

DNA arrays are solid supports upon which a collection of gene-specific nucleic acids have been placed at defined locations. In array analysis, a nucleic acid-containing sample is labelled and then allowed to hybridise with the gene-specific targets on the array. Based on the amount of nucleic acid from the sample hybridised to target on the array, information is gained about the specific nucleic acid composition of the sample. Array analysis, according to the present invention, involves isolating total RNA from a sample comprising cells or microvesicular material, converting the RNA samples to labelled cDNA via a reverse transcription step, hybridising the labelled cDNA to identical arrays (such as via either a nylon membrane or glass slide solid support), removing any unhybridised cDNA, detecting and quantitating the hybridised cDNA, and determining the quantitative data (e.g. the levels of biomarkers present) from the various samples.

Real-time or quantitative PCR refers to a method which monitors the replication of a nucleotide sample in real-time during the PCR reaction. As well as the normal components, the reaction mixture contains fluorescent probes which may hybridise to any double-stranded nucleotide sequence or else to a specifically chosen complementary sequence. The signal from the fluorescent probes therefore correlates with the number of the target sequences which have been produced during the reaction and can be used to determine the quantity of the target sequence in the original sample.

Analysis of RNA levels (e.g. quantifying the RNA concentration or amount) within the liquid biopsy sample allows for adjustment of the generic settings (standard baseline settings) for the simulation algorithm to correspond to those of the individual subject. In this regard the methods of the invention allow for the determination of the biomarker profile generated for the individual to be correlated with the corresponding levels of drug metabolizing, transporting and clearance proteins (e.g. ADME proteins) within the tissues of said individual, such as in the liver, kidney, CNS or gut and on the surface of the target cell. This step comprises quantifying the levels (including concentration/amount/activity) of at least one biomarker within the sample from the individual which is correlated to at least one level of a drug metabolizing, transporting and clearance protein or enzyme within the individual's tissues via a defined correlation function or algorithm. Hence, the model according to the invention is able to determine a correlation between first input data in the form of a biomarker profile from the sample and the activity/concentration/amount level of drug metabolizing, transporting and clearance protein or enzyme within the individual's tissues. This enables the production of an augmented profile that comprises baseline biomarker data from the original sample together with the correlated (or predicted) level of drug metabolizing, transporting and clearance protein or enzyme within the individual's tissues. The augmented first input data is used to define the starting points for the simulation model of the invention, in terms of baseline levels of drug metabolizing, transporting and clearance protein or enzyme within the individual's tissues, optionally in combination with gene identity and expression data including allelic variation and whether certain genes are either up- or down-regulated compared to average (i.e. mean or median) levels within a given population. The term “down-regulated” as used herein denotes a process resulting in decreased expression of one or more genes and/or the proteins encoded by those genes. “Up-regulated” denotes an increase in gene expression and corresponding protein expression.

Additional factors may have a bearing on ADME protein abundance. These characteristics may be determined by the measurement of biomarkers in a sample, which can be the same or different sample as the liquid biopsy sample used for determination of the one or more mRNAs coding for drug metabolizing enzymes or drug clearance proteins. For example, allelic variations of CYPs, transporter genes or transferases, or any other relevant gene, may be determined from genomic DNA isolated from a liquid biopsy sample or any of a number of biological samples. This can include information not able to be derived from mRNA sequences, such as intron data, epigenetic information and the presence and activity of genomic regulatory features such as promoters, repressors, and so on.

Non-gene expression parameters which may also be relevant for determining health status in an individual subject may include parameters which can be determined by measurement of biomarkers in one or more liquid biopsy sample, and/or can include physiological and epidemiological information collected by other means. In some embodiments of any aspects of the invention, one or more non-gene expression parameters may be selected from the group consisting of: ethnicity; genotype; age; age group classification; gender; smoking status; presence of chronic disease, including renal impairment, diabetes (type I or type II) or liver cirrhosis; body mass index (BMI); body adiposity index (BAI) or other equivalent measurements of body fat content; waist circumference measurement; waist-to-hip ratio; hydrostatic weighting; average alcohol consumption; pregnancy; allergy status; blood pressure; total blood lipids (e.g. cholesterol); average resting heartbeat; ECG interval measurements including QT interval, QRS duration, and PR intervals; general medical history; familial medical history; or combinations thereof. Such additional parameters may be used to further refine any model, algorithm, simulation or prediction produced by the invention, improving accuracy.

Embodiments of the present invention provide a method that is used to build a robust computer (in silico) predictive model of drug metabolism, in particular drug distribution and clearance, for a specified individual subject. In this way a computer-based model of drug clearance can be matched to any given individual, following a simple blood test, and thereby provides an accurate personal prediction of an individual's capacity to metabolize and/or clear a given drug, xenobiotic, or combination of drugs or xenobiotics. The so-called Virtual Twin model is incorporated into a computer implemented system that can be utilised by, for example, clinicians, academics, patients and pharmaceutical researchers.

According to an embodiment of the invention the method comprises the steps of obtaining a liquid biopsy sample from an individual. The liquid biopsy may suitably comprise a bodily fluid such as any one or more of: blood, urine, saliva, semen, tears, lymphatic fluid, cerebrospinal fluid, bile, stool or a mucus secretion. This sample can be obtained via a minimally invasive route, and can include deriving blood components such as plasma, serum or other sample from a whole blood liquid biopsy sample. The sample is analysed quantitatively to determine the levels of one or more, typically a plurality, of mRNAs coding for drug metabolizing enzymes or drug clearance proteins (e.g. ADME proteins) in order to derive a profile of the said individual's circulating mRNA. The sample is also analysed to determine the levels of one or more organ specific marker genes in the sample in order to give a subject-specific organ shedding correction factor (SCF). The SCF is used to provide a baseline for the rate or amount of circulating mRNA shed by the organ and is thus used as an adjustment factor in order to correct the profile of the individual's circulating mRNA.

The corrected profile defines first input data, which first input data may then be used to inform a computer-based (in silico) model of ADME protein abundance within the body of the subject. The calibration step thereby enables the creation of an individual model. This individual model accurately predicts organ function, health status and may also be used to assess the pharmacokinetics and pharmacodynamics of drug metabolism and clearance, for a specified xenobiotic or pharmaceutical compound or combination of the same. Hence, the invention provides a robust model for a specified subject that may be interrogated by clinicians, researchers and artificial intelligence algorithms.

In some embodiments the method further comprises quantitatively analysing a sample to determine the levels of one or more, typically a plurality, of biomarkers present within the sample in order to derive a profile of the said individual's biomarker(s). The sample may be the same or different to that sample for determining circulating RNA, and as such may further include the steps of obtaining a second biological sample from an individual. The sample may be obtained in any suitable way, but may again be obtained via a minimally invasive route, such as a blood, cheek swab, saliva, stool or urine sample. The profile defines biomarker input data, which biomarker input data is then used to further calibrate the computer-based model of drug clearance.

In some embodiments, physiological and/or epidemiological information to obtain non-gene expression data not derivable from sample biomarkers may be obtained from an individual, in order to derive a physiological and/or epidemiological profile of the said individual. Such information may include ethnicity; age; gender; smoking status; body mass index (BMI); body adiposity index (BAI) or other equivalent measurements of body fat content; waist circumference measurement; waist-to-hip ratio; allergy status; blood pressure; average resting heartbeat; ECG interval measurements including QT interval, QRS duration, and PR intervals; general medical history; familial medical history; or combinations thereof. The profile defines personal input data, which is then used to further calibrate the computer-based model of drug clearance.

One embodiment of the present invention provides a sophisticated platform for the analysis of pharmacokinetic outcomes, drug-drug interactions (so-called DDIs) and tissue-specific responses in a given individual, resulting in a comprehensive physiologically-based pharmacokinetic (PBPK) model. PBPK models can comprise nested compartments that represent different tissue functionalities and cell types within an organ system. When assembled, the levels of hierarchical complexity allow for modeling of molecularly-driven events, such as specific metabolic pathways. The blood flows and partition coefficients that link the compartments—i.e. the organ systems—together mathematically are estimated from animal, in vitro data, and clinical data. The parameters and compartments are then optimized to fit the model to existing data.

Hence, the present invention provides a significant advantage over and enhancement of prior art modelling systems that are largely based upon population level, animal or entirely in vitro based responses. In contrast, according to specific embodiments the present invention provides a virtual mimic, also referred to as a “Virtual Twin”, for an individual. This Virtual Twin may represent an in silico model that is configured so as to represent an entirely personalised PBPK model for a given individual. The model may represent the consolidation of multiple data inputs from a variety of sources, including the physiological and/or epidemiological information described above, the genotype as well as a SCF. This approach facilitates the growth of personalised medicine solutions, improved design of dosage regimens and the identification of potentially harmful side effects before a drug, xenobiotic, or combination of same is administered. In addition the present invention may provide a direct correlation between the levels of circulating RNA present in, for example, the blood with the level (e.g. abundance) of ADME proteins, drug metabolizing, transporting and clearance proteins within tissues such as the liver in that individual. Previous approaches have only looked to correlate mRNA and/or biomarker levels with estimates of enzyme activity against a specified probe compound, and as a result have struggled to gain meaningful traction outside of the very limited probe-enzyme system described.

The virtual simulator may also incorporate an in vitro to in vivo extrapolation (IVIVE) approach to further inform the model. The IVIVE approach establishes virtual populations by building up mechanistic and physiologically based pharmacokinetic (PBPK) models. These models incorporate identified variabilities in demographic and biological (genetic and environmental) components linked to drug-specific physicochemical properties (for example, aqueous and lipid solubilities) and in vitro data on absorption, metabolism and transport. The covariate relationships embedded in such models can be complex and nonlinear and can be difficult to resolve by simple linear covariate analysis. The primary advantage of the IVIVE approach is that it maximizes the value of all in vitro information previously generated during drug discovery and preclinical development.

The algorithm of an embodiment of the invention may include consideration of SCF-corrected input data comprising data pairs or even data clusters. Suitably, data derived from mRNA analysis, such as gene expression data for drug clearance genes, may be categorised further via one or more additional gene and non-gene expression parameters, which may be derived from analysis of biomarkers detected in one or more biological samples. Non-gene expression parameters may include physiological and epidemiological information. In some embodiments of any aspects of the invention, one or more non-gene expression parameters may be selected from the group consisting of: ethnicity; genotype; age; age group classification; gender; smoking status; presence of chronic disease, including renal impairment, diabetes or liver cirrhosis; body mass index (BMI); body adiposity index (BAI) or other equivalent measurements of body fat content; waist circumference measurement; waist-to-hip ratio; hydrostatic weighting; average alcohol consumption; pregnancy; allergy status; blood pressure; total blood lipids (e.g. cholesterol); average resting heartbeat; ECG interval measurements including QT interval, QRS duration, and PR intervals; or combinations thereof.

In a specific embodiment of the invention, the described methods can be implemented via one or more computer systems. According to a further embodiment, an apparatus comprising one or more memories and one or more processors is provided, wherein the one or more memories and the one or more processors are in electronic communication with each other, the one or more memories tangibly encoding a set of instructions for implementing the described methods of the invention. In another embodiment the invention provides a computer readable medium containing program instructions for implementing the method of the invention, wherein execution of the program instructions by a controller comprising one or more processors of a computer system causes the one or more processors to carry out the steps as described herein. Suitably, the data may be stored in a database, and accessed via a server. Suitably, the server is provided with communication modules to receive and send information, and processing modules to carry out the steps described herein. In some embodiments, the data is provided through a cloud service. In preferred embodiments, the method is accessible as a web service. In some embodiments, users may access the service for recordal or retrieval of scores via a website, in a browser. Networking of computers permits various aspects of the invention to be carried out, stored in, and shared amongst one or more computer systems locally and at remote sites. Hence, two or more computer systems may be linked using wired or wireless means and may communicate with one another or with other computer systems directly and/or using a publicly-available networking system such as the Internet.

Suitably, the computer system includes at least: an input device, an output device, a storage medium, and a microprocessor. Possible input devices include a keyboard, a computer mouse, a touch screen, and the like. Output devices computer monitor, a liquid-crystal display (LCD), light emitting diode (LED or OLED) computer monitor, virtual reality (VR) headset and the like. In addition, information can be output to a user, a user interface device (e.g. tablet PC, mobile phone), a computer-readable storage medium, or another local or networked computer. Storage media include various types of memory such as a hard disk, RAM, flash memory, and other magnetic, optical, physical, or electronic memory devices. The microprocessor is a computer microprocessor (e.g. CPU) for performing calculations and directing other functions for performing input, output, calculation, and display of data. The microprocessor may serve to direct and control the computer system of embodiments of the invention. In one embodiment of the invention, the computer processor may comprise an artificial neural network (ANN). In a further embodiment of the invention the computer processor may comprise a machine learning algorithm, suitably a machine learning algorithm that has been trained against one or more appropriate data sets.

A simulation algorithm of an embodiment of the invention handles a multiplicity of pharmacokinetic (PK) model combinations, including:
(1) Administration of single (small and large molecules) or multiple chemical moieties,
(2) Different absorption models, namely one-compartment, enhanced Compartmental Absorption and Transit (CAT), and Advanced Dissolution, Absorption and Metabolism (ADAM) models,
(3) Different distribution models such as minimal and full PBPK models with different perfusion- and permeability-limited models, including multi-compartment organ, kidney, blood-brain-barrier, intestinal degradation models and an additional multi-compartment user defined organ/tissue,
(4) Modelling of a plurality of metabolites, and
(5) Pathology or disease state of the subject or population being modelled, e.g. whether the subject or population are healthy or not.

According to one embodiment of the invention, PBPK model algorithms are built using ordinary differential equations (ODE) (for example see Jamei M et al., Expert Opin Drug Metab Toxicol. 2009 February; 5(2):211-23; and Jamei et al. AAPS J. 2009 June; 11(2): 225-237; Nagar et al. Mol Pharm. 2017 Sep. 5; 14(9): 3069-3086).

The methods of the invention are particularly useful in contributing to improved construction of PBPK models by providing better understanding over how abundance of drug clearance proteins can vary between individuals. This is important as a result of the increased dependency on PBPK models to address regulatory questions as well as their ability to minimize ethical and technical difficulties associated with pharmacokinetic and toxicology experiments for special patient populations. Hence, the invention provides, in one embodiment, an improved method for creation of computer based models for the determination of clearance of a given xenobiotic molecule (e.g. drug or biological therapeutic) from individual, or when cumulative data is provided, from a population of individuals. The ease of liquid biopsy, which is far less invasive then solid tissue biopsy sampling, is a major factor in contributing to improved construction of computer models that show utility in drug development and clinical trial design. It also enables new models to be created for use in distinct cohorts such as for neonatal and paediatrics as well as in smaller ethnically distinct populations, or for rare diseases, by way of non-limiting example.

The exposure of an individual to a certain drug can be measured by the area under the concentration time curve (AUC). The AUC after administration through any non-parenteral route (such as an oral dose) is dependent on the proportion of the dose that is absorbed and is subsequently available in the systemic circulation. In the case of oral drug administration (the most common route for drug intake), this involves release of the drug from the formulation, passage through the gut wall and then through the liver. The bioavailability of the drug (F) together with the clearance (CL) and the dose of the drug (D) will determine the overall exposure (AUC) according to the following equation 1 below:

$$AUC = \frac{F \times Dose}{CL}$$

Total clearance (CL) is defined as the volume of blood completely cleared of drug per unit time and encompasses clearance by the liver, the kidneys and biliary excretion (in the absence of re-absorption from the gut). Although exposure to the drug is determined only by the dose, clearance and bioavailability, varying shapes of concentration-time profile can occur for a given exposure when the rate of entry (absorption rate, infusion rate etc.) and rate of elimination are changed. Elimination rate is a function of clearance and distribution characteristics.

Since the majority of drugs currently on the market are lipophilic, metabolism is a major route of elimination from the body. It should be noted that overall metabolic clearance is not usually a simple linear function of the organ but it is also dependent on the delivery of the free drug to the site of metabolism. By way of example: hepatic clearance, distribution and metabolism may be determined by factors such as hepatic blood flow, plasma protein and red blood cell binding, and the effects of influx into or efflux from hepatocytes. In vivo intrinsic organ clearance has been extrapolated from in vitro models using human liver microsomes/exosomes or human hepatocytes in culture. However, to determine whole organ or even systemic clearance requires the combination of intrinsic clearance rates for multiple drug clearance/metabolising enzymes and transporters in different organs and tissues. For each individual the levels of these enzymes will vary, thus resulting in a different level of clearance for that individual.

An expression that estimates the net intrinsic metabolic clearance in total by the whole liver (CLu_{H,int}) from data obtained with recombinantly expressed CYP enzymes is given by equation 2, below:

$$[ {\sum }_{j = 1}^{n} ( {\sum }_{i = 1}^{n} {ISEF}_{j i} \times \frac{{V}_{maxi} ( {rhCYP}_{i} ) \times {CYP}_{j} abundance}{{K}_{mi} ( {rhCYP}_{j} )} ) ] \times MPPGL \times Liver weight$$

where there are i metabolic pathways for each of j CYPs, rh indicates recombinantly expressed enzyme, Vmax is the maximum rate of metabolism by an individual CYP, Km is the Michaelis constant, MPPGL is the amount of microsomal protein per gram of liver and ISEF is a scaling factor that compensates for any difference in the activity per unit of enzyme between recombinant systems and hepatic enzymes. This expression indicates that inter-individual variability in hepatic intrinsic clearance can be introduced by incorporating variability in several important parameters. One key parameter influencing this model is the liver abundance (e.g. the level) of each CYP in the individual. Other parameters such as MPPGL and liver weight can be estimated based upon height, weight and age of the individual. However, it is the differences in in metabolism that result from CYP abundance, as well as functional, genetic polymorphisms that can be accommodated by knowing the frequency of different genotypes, and by modifying either the enzyme abundance or the intrinsic enzyme activity. Data on changes in the abundance and/or activity of different xenobiotic clearance proteins, such as CYPs, is incorporated into the virtual model of the present invention in order to predict hepatic clearance in individuals.

Hence, the virtual in silico model of an embodiment of the invention comprises algorithms that are able to incorporate in vitro data on drug metabolism/clearance and inter-individual variability that is relevant to drug metabolism/clearance in the tissues of the individual concerned. Optionally, the virtual model of the invention may further incorporate allometric scaling models. Allometric scaling methodology attempts to predict mean clearance values in humans from those observed in animal species by scaling for body size. The use of an approach that incorporates IVIVE in addition to allometric scaling has the added advantage of being able to assess the likely individual allelic variability in clearance. For example, some allelic variations of CYP enzymes show decreased catalytic activity compared to wild type and, thus, having knowledge of an individual's specific genotype enables in vitro data on kinetics to be used to estimate in vivo clearance.

Creating a simulator that provides accurate clearance and/or metabolic capacity values for an individual requires the consideration of multiple parameters—amongst other things: size of organ, genotype of certain enzymes, kidney function etc. However, of critical importance is the affinity of a given xenobiotic compound (e.g. drug) for the drug clearance proteins present and efficiency of each molecule of protein in handling each molecule of xenobiotic compound. This relationship is described as the K_{cat} and is based on intrinsic clearance of the drug by a given enzyme. By way of example, as described previously, certain drugs are metabolized by particular CYPs, transferases and operate through specific membrane transporters, as well as specific combinations of these drug clearance proteins. The K_{cat} for each CYP or transferase with a given drug is a key determining factor ascertained during clinical trials of any pharmaceutical compound. Hence, if the K_{cat} and/or CLᵢₙₜ is a key parameter that is determined in all modern drug development programs, then by estimating the abundance of the relevant drug clearance proteins in an individual's organ, such as the liver, it is possible to predict the clearance of that drug in that individual. The virtual simulator, of one embodiment of the invention, operates by summing up the various clearances and putting them through the appropriate models of the organ, that consider inter alia the limitations of blood flow and availability of free drug concentration in the blood to the organ. Previous attempts at creating such models have typically resulted in low success because they relied upon unmatched samples or estimates of activity based upon literature referenced population averaged levels of abundance.

Accordingly, in a specific embodiment of the invention a dosage regimen is provided, in which parameters related to the administration of a drug comprising a pharmaceutical compound or a biological therapeutic agent to a subject are determined in conjunction with that individual's clearance capacity for the compound or agent as well as their health status. More specifically, a liquid biopsy may be obtained from a subject and cfRNA_{TOTAL} analysis performed. From the cfRNA_{TOTAL} analysis a SCF for the subject can be determined. PBPK and popPK clearance models are understood for a wide range of approved pharmaceutical compounds and compound classes. In particular, the specific drug metabolising CYPs, transferases and transporters etc. that are relevant for clearance of most compounds and agents and, therefore, the one or more clearance proteins that constitute [cfRNA]_{Target} for the given drug may be identified. The normalized [cfRNA]_{Target} for the specific clearance protein(s) may be determined from the cfRNA_{TOTAL} thereby enabling the abundance of the specific ADME protein(s) within the relevant organs of a given individual to be ascertained. It will be appreciated that having information of this type for any given individual in relation to a proposed a pharmaceutical compound or a biological therapeutic agent that is to be administered enables the health status a precision dosing regimen to be formulated for that individual for the specific drug.

Reporting of the output data from the system of the invention may be achieved via the GUI or via an output file that may comprise a .csv file or spreadsheet, such as Microsoft Excel™ (Microsoft Corp., Redmond (Wash.), USA) or Google Sheets (Google LLC., Mountain View (Calif.), USA). By way of non-limiting example, when the reporting process is implemented through the Excel Automation interface which is based on the Office Object Model. The simulation platform uses this technology to create or connect to an Excel application Component Object Model (COM) object, to manipulate and add worksheets as required. Each worksheet is a bespoke output based on the simulation input selections: each cell is effectively created individually with the selection of font (including size and weight), colour (both foreground and background), alignment of text within the cell, number format (based on the users' machine selection) as well as many other specifications.

After the output data has been rendered, graphical representations, such as dashboards, charts, pictograms or graphs may are added if applicable. These may include concentration-time profiles or, for example, pie charts of enzyme contribution, ratios of key ADME protein levels within the tissue of the subject, all of which are created based on the output data comprised within the worksheet and formatted individually based on user selections such as number format, dashboard arrangement and also the colour ‘skin’ chosen before displaying the data.

In an alternative embodiment of the invention output data is comprised within a relational database. An advantage of this embodiment is that the simulator algorithm may be comprised as part of an organisational workflow as it can then write directly into a corporate database, for example. This enables formatting and visualisation and data analytics to be customised by the user.

Embodiments of the invention may also relate to an apparatus or device for performing a set of operations as defined herein, such as a set of operations that may suitably implement at least one embodiment of the present invention. The apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of medium suitable for storing electronic instructions, which may be coupled to a computer system bus. Furthermore, any computing systems referred to in the specification may include a single processor or may be architectures employing multiple processor designs for increased computing capability.

Any of the steps, operations, methods or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, methods or processes described.

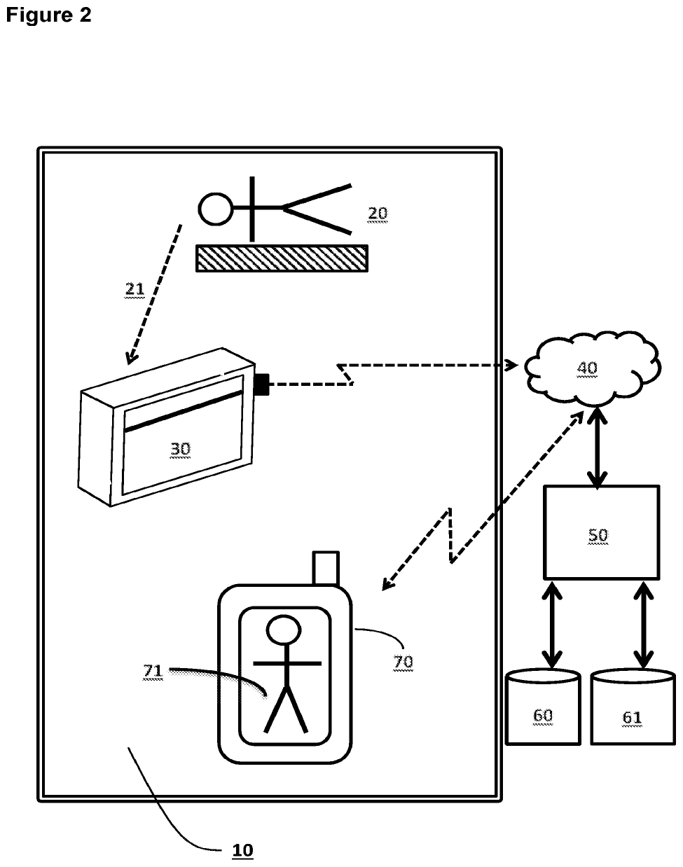

One embodiment set out in FIG. 2 provides a system for generation of a virtual model of organ or tissue health in a subject 20 who may be an asymptomatic individual or a unwell patient in need of a therapy. The subject 20 may attend a testing facility 10 directly as shown, or may be tested remotely from home, in the field or in a clinical setting such as a health centre, doctor's surgery or hospital. For convenience the system is shown as occurring within a testing facility 10, although it is appreciated that this is non-limiting. A liquid biopsy 21 is taken from the individual subject 20. The biopsy 21 may comprise a sample of blood, but may also comprise any predominantly liquid sample material that comprises cell free organ mRNA. The liquid biopsy 21 is subjected to an automated RNAomics analysis 30. Once again, for convenience the analysis is shown as occurring within the testing facility 10, although it will be appreciated that remote testing via an analytics service is also a possibility. The result of the analysis 30 is communicated securely either directly or via the cloud 40 to one or more remote servers 50. The server 50 may perform computational analysis according the aforementioned methods in order to provide a quantitative output of the biopsy 21 which establishing the levels—or abundance—of ADME proteins in an organ(s) of the subject 20. The server 50 may communicate with further servers 60,61 that host additional in silico modelling capacity (such as PBPK modelling—for example, SimCyp™ Simulator: www.certara.com) enabling generation of a more sophisticated virtual model of tissue functioning status 71 of the subject 20. Communication of this model 71 to one or more user interface/output devices 70 allows for local interrogation by a clinician or scientist with proximity to the subject 20, or even by the subject 20 themselves, where data protection and ethics regulations permit. The model 71 may be provided via the output device 70 in the form of a simulation, or via an automation interface that allows for data display and interrogation. Suitable outputs may include spreadsheets, charts, graphs, tables, figures and such like. Clinical decisions, such as diagnostic predictions and/or consideration of suitable treatment options may be based upon the output of the device.

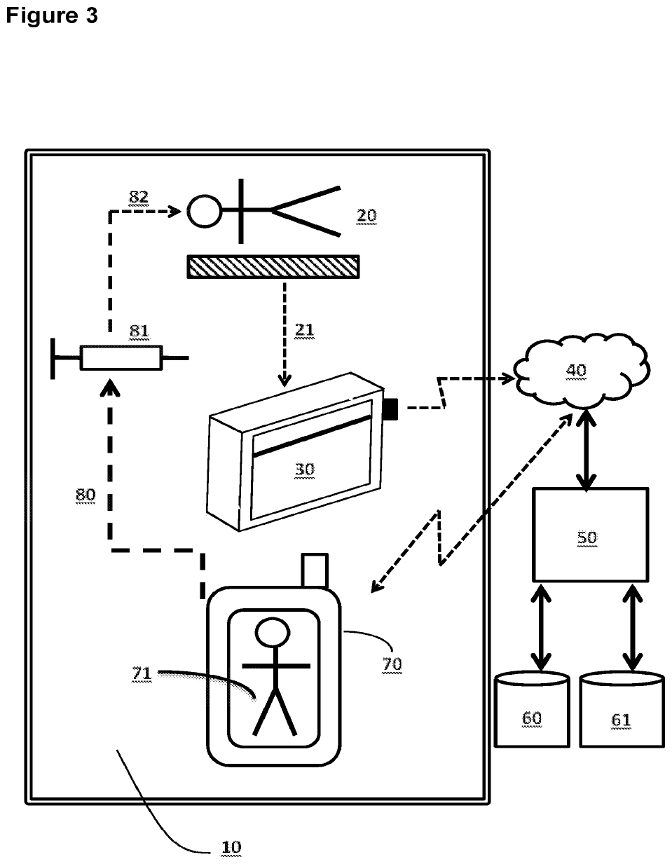

A further embodiment in FIG. 3 is shown, in which the model 71 is used as an input to inform a dosage regimen 80 that is applied to a drug or other therapeutic agent, or combination of agents, 81. The drug 81 is then administered to the subject 20, in accordance with the dosage regimen 80. Optionally, additional liquid biopsy 21 may be taken following administration of the drug 81 in order to monitor a number of parameters that may be pertinent to updating of the model 71 and/or refinement of the regimen 80 going forward. The dosage regimen may be varied according to the needs of the patient as determined via the model 71, for example over time or in relation to changing health status as determined according to the methods of the invention. Hence, in an embodiment of the invention there is provided a method of treating a patient over a period of time. The time may be measured in hours (e.g. at least one hour and at most 24 hours) if acute therapy is required, or over days (e.g. at least one day and at most seven days), weeks (e.g. at least one week and at most six weeks), months (e.g. at least one month and at most twelve months) or years (e.g. at least one year and at most five years or more) in the case of chronic conditions or exposure.

In embodiments of the invention, dosage regimens and improved personalised methods of treatment for specific classes of drugs may be optimised. Suitably, classes of drugs may include any one of the group consisting of: an anti-inflammatory drug; an anti-cancer drug; an anti-biotic; an antiviral; an anti-fungal; an analgesic; an anaesthetic; an anti-allergenic; an antidote; a hormone replacement drug; an immunosuppressive; an anti-coagulant; a cardiovascular drug; an anti-depressant; an anti-diabetic; an anti-psychotic; a diuretic; a vitamin; and a sedative. In a specific embodiment of the invention, the dosage regime and/or improved personalised method of treatment is generated for any one or more of the products appearing on the World Health Organisation Model List of Essential Medicines (20th Edition, amended August 2017; http://www.who.int/medicines/publications/essentialmedicines/en/), which is incorporated herein by reference. In alternative embodiments, dosage regimens for any one of the pharmaceutical compounds recited above, as well as their salts, is provided for according to the methods of the present invention.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

The invention is illustrated by the following non-limiting examples.

### EXAMPLES

### Example 1

The following example provides a protocol for total RNA extraction from samples of blood that can be used to determine the levels of RNA for drug metabolizing enzymes, transporters and/or marker genes in the samples, and/or RNA for the determination of biomarkers. Methods for the isolation of total protein and quantification of enzymes and transporters are described herein for the assessment of correlation between plasma RNA and tissue protein levels.

RNA Analysis of Liquid Biopsy Comprising Blood

A. Blood Samples:

A liquid biopsy consisting of fresh peripheral venous blood may be collected from a subject and plasma isolated before further processing as described below. If required, peripheral blood mononuclear cells (PBMCs), including B and T lymphocytes, may be isolated using Ficoll-Paque PLUS (GE Healthcare Life Sciences).

Isolated plasma is stored frozen −80° C. until used for cell free RNA (cfRNA) isolation and measurement. Isolation of circulating or exosomal RNA can be done using a suitable RNA extraction kit such as the Qiagen QIAamp Circulating Nucleic Acid Kit as per the manufacturer's instructions (Qiagen, Hilden, Germany). Total nucleic acid is collected by such kits, and DNA is removed using a suitable kit such as the Qiagen RNase-free DNase Set or Ambion Turbo DNA-free Kit (Life Technologies, Carlsbad, Calif., USA). Eluted RNA, after DNA removal, is then detected as a quality control using a suitable total nucleic acid assessment technique such as the Agilent RNA pico Kit on Bioanalyzer equipment (Agilent Technologies, Eugene, Oreg., USA). RNA of sufficient quality is then stored for subsequent quantification.

B. Reverse Transcription-PCR and Gene Sequencing:

RNA (5-10 ng) may be reverse-transcribed using M-MLV Reverse Transcriptase (Invitrogen, Life Technologies, Inc.). Samples are amplified with PCR in a final reaction volume of 25 μl containing 2.5 μl of 10 times buffer, 0.1 μl of 10 mM dNTPs, 10 pmoles of each primer and 0.5 units of Taq DNA Polymerase. To confirm the presence and integrity of the cDNA template, the housekeeping gene, GAPDH, is amplified for each sample using primers GAPDH-5 (5′-ACCACAGTCCATGCCATCAC-3′; SEQ ID NO: 1) and GAPDH-3 (5′-TCCACCACCCTGTTGCTGTA-3′; SEQ ID NO: 2). Conditions may be as follows: an initial denaturation step for 5 minutes at 94° C., then 50 seconds at 94° C., 45 seconds at 55° C., and 1 min at 72° C. for 30 cycles, followed by an elongation step for 10 minutes at 72° C.

The cDNA obtained from the extracted total RNA may be analysed further, such as via a DNA microarray, in order to determine the identities and expression levels of genes expressed within the PBMCs and the tissue biopsy samples. Alternatively, reverse transcription and amplification can be performed using a suitable genome sequencing method, such as Ampliseq (Life Technologies, ThermoFisher, Austin, Tex.) or NextSeq 550Dx (Illumina, USA). Over 20,000 genes can be sequenced and several libraries (one library per sample) can be analysed in one experiment. The level of achievable amplification may depend on the quality of the isolated RNA material and the required depth of analysis. As an example, determination of the expression of the following cytochrome P450 mono-oxygenase genes linked to drug and xenobiotic compound metabolism may be determined in both the plasma as well as in the organ samples: CYP1A1; CYP1A2; CYP1B1; CYP2A6; CYP2A7, CYP2A13; CYP2B6; CYP2C8; CYP2C9; CYP2C18; CYP2C19; CYP2D6; CYP2E1; CYP3A4; CYP3A5 and CYP3A7. Other genes which may be determined include marker genes for hepatocytes, in order to determine the SCF for the particular individual (see Example 2, below).

The above protocol may be repeated as necessary for multiple individuals in order to generate data on expression levels of genes linked to drug and xenobiotic compound metabolism and/or the expression of organ marker genes. The data is suitable for interrogation via bioinformatics techniques to determine correlations between marker expression in circulating mRNA and expression of CYPs, for example, in the organ sample. The correlations are used to develop a virtual model of xenobiotic compound clearance that can be configured on a person by person basis in order to provide a virtual twin model of compound clearance within a given individual.

### Example 2

The following example provides a protocol for determining the degree of RNA shedding into circulation from hepatocytes in a particular subject, so establishing a robust and significant correlation function between hepatic protein levels and the corresponding plasma RNA concentrations.

Marker genes: A1BG (Alpha-1-B glycoprotein), AHSG (alpha-2-HS-glycoprotein), ALB (Albumin), APOA2 (Apolipoprotein A-II), C9 (Complement component 9), CFHR2 (Complement factor H-related 5), F2 (Coagulation factor II (thrombin)), F9 (Coagulation factor IX), HPX (Hemopexin), SPP2 (Secreted phosphoprotein 2), TF (Transferrin), MBL2 (mannose-binding lectin (protein C) 2); SERPINC1 (Serpin peptidase inhibitor, clade C (antithrombin), member 1) and FGB (Fibrinogen beta chain).

The use of an SCF based on the 13 selected genes reduces the effects of technical variability inherent to using only one gene, such as albumin (ALB), as a reference. It is known that the level of shedding in cancer patients can be severalfold higher and can be more variable than that in healthy controls. Without wishing to be bound by theory, the increased amount of RNA shedding (and observed larger variability) in cancer patients may result from cell death (necrosis), possibly also in response to chemotherapy. Nevertheless, the identification of this phenomenon permits correction and accommodation within models generated by the methods of the invention.

Therefore, due to the presence of different levels of RNA shedding amongst test subjects, the correction being applied to enzyme expression levels in plasma is as follows (e.g. using 13 markers).

$$SCF = 1 {0}^{6} \cdot {\sum }_{i = 1}^{13} {[ cfRNA ]}_{{Marker}_{i}} / ( 12 \times {[ cfRNA ]}_{TOTAL} )$$

$${( {[ cfRNA ]}_{Enzyme} )}_{Normalized} = {[ cfRNA ]}_{Enzyme} / SCF$$

[cfRNA]_{TOTAL} is the total RNA reads in a library generated from the plasma sample of Experiment 1. The outcome should be a normalized reading for each enzyme expressed out of a million reads in a plasma sample of specified volume (e.g. 1-5 ml).

### Example 3

Quantification of Drug Metabolizing Enzymes in Liver from SCF Adjusted CYP cfRNA Levels Determined from Liquid Biopsy

The amounts of circulating plasma mRNA can be used to identify the relative abundance of a plurality of hepatic proteins that control xenobiotic compound clearance. Table 1 shows examples of abundance values that permit such estimation for four specific drug clearance enzymes in the liver of human subjects based upon the SCF-adjusted plasma concentration of the corresponding mRNA (i.e. [CYPnnn]ₚₗₐₛₘₐ]).

TABLE 1
Liver protein abundance equations
from circulating RNA measurements

| Enzyme | Equation |
|---|---|
| CYP3A4 | [CYP3A4]ₜᵢₛₛᵤₑ = 29.68 × [CYP3A4]ₚₗₐₛₘₐ + 0.62 |
| CYP2C9 | [CYP2C9]ₜᵢₛₛᵤₑ = 17.16 × [CYP2C9]ₚₗₐₛₘₐ + 0.46 |
| CYP1A2 | [CYP1A2]ₜᵢₛₛᵤₑ = 0.43 × [CYP1A2]ₚₗₐₛₘₐ + 0.18 |
| CYP2A6 | [CYP2A6]ₜᵢₛₛᵤₑ = 7.54 × [CYP2A6]ₚₗₐₛₘₐ + 0.54 |
| CYP2C19 | [CYP2C19]ₜᵢₛₛᵤₑ = 0.75 × [CYP2C19]ₚₗₐₛₘₐ − 0.06 |
| CYP2D6 | [CYP2D6]ₜᵢₛₛᵤₑ = 7.54 × [CYP2D6]ₚₗₐₛₘₐ − 0.29 |

As mentioned previously the abundance of the enzyme in the liver is capable of being correlated to a biological activity for that enzyme, thereby allowing for a range of parameters to be calculated including net intrinsic metabolic clearance as absolute values as well as ratios of CYPs relative to each other or to other ADME proteins. These latter parameters may have significant prognostic and diagnostic value.

### Example 4

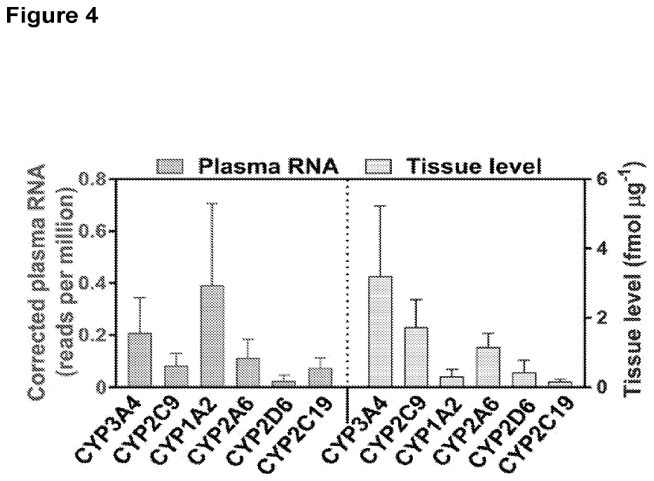

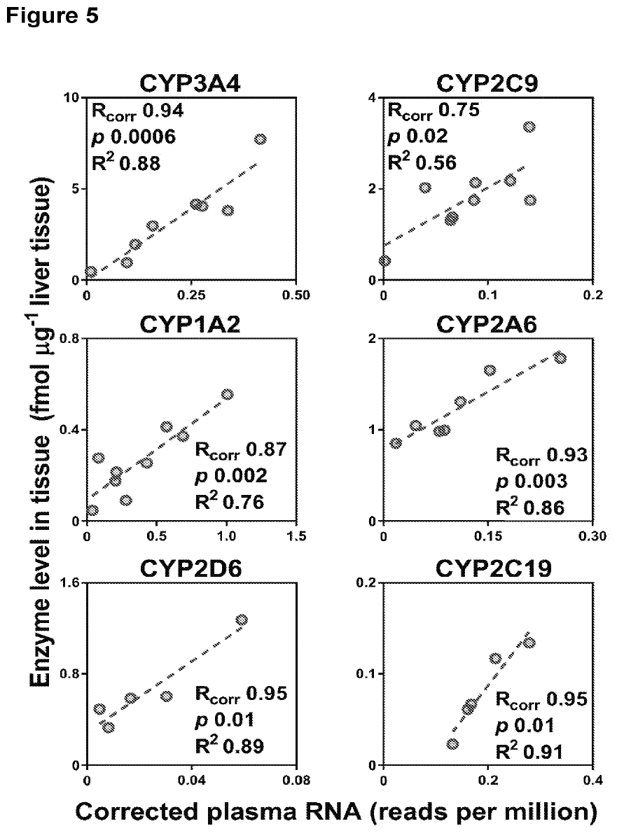

Equivalent approaches were repeated on a different range of matched samples of liver tissue with corresponding blood samples. Next generation sequencing, in this instance using an Illumina preparation and analysis protocol, was performed on purified exosomal RNA obtained from blood samples for six CYPS and correlated to the corresponding concentration in the tissue according the techniques described herein. The results are shown in FIG. 4 shows the plasma concentration for the mRNA for each CYP following shedding correction and the corresponding tissue levels (in fmol μg⁻¹) of the same proteins—where n is equal to the number of samples processed. The relationship between the adjusted plasma mRNA values and the corresponding enzyme concentrations in the tissue of origin is set out in FIG. 5. It is evident the high level of correspondence seen allows for accurate determinations of tissue protein concentration only after adjustment for shedding correction is made.

The approach taken in the present invention allows for the creation of in silico models for individuals and populations that will permit the more accurate predictive modelling of health status within the tissue of origin. In the absence of the shedding correction the results are highly variable between individuals rendering liquid biopsy analysis impractical and inaccurate.

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid starting material, the clone of interest, or type of library used is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1. A system for assessing the status of the health of a tissue within the body of a subject, the system comprising:
a receptacle for receiving a liquid biopsy sample obtained from the subject, wherein the liquid biopsy sample comprises at least a first cell free mRNA (cf-mRNA) that encodes a first target protein, and wherein the first target protein is a protein that is expressed in the tissue and the concentration of the first target protein in the tissue is indicative of the health status of the tissue;
a process module for processing the liquid biopsy sample to quantify the amount of first cf-mRNA present, wherein the process module comprises liquid handling apparatus for isolating and quantifying total cf-mRNA from the liquid biopsy sample; and
at least one analytics module for determining the concentration of the first cf-mRNA in the liquid biopsy sample, wherein the analytics module is in communication with the process module, the at least one analytics module comprising at least one controller wherein the controller performs a normalization function in order to correct for mRNA shedding in the subject, and the normalized concentration of the first cf-mRNA is further associated by the controller to the abundance of the first target protein in the tissue by a standard correlation function;
an interface module for providing an output to a user of the system, wherein the interface module provides a categorization of the health of the tissue within the body of the subject.

2. The system of claim 1, wherein the tissue is comprised within the liver of the subject.

3. The system of claim 1, wherein the disease is selected from the group consisting of: fatty liver; non-alcoholic steatohepatitis (NASH); cirrhosis; liver disease; hepatitis and liver cancer; and a combination thereof.

4. The system of claim 1, wherein the disease is NASH.

5. The system of claim 1, wherein the disease is cirrhosis of the liver.

6. The system of claim 1, wherein the liquid biopsy comprises a sample of a bodily fluid selected from the group consisting of: blood; urine; saliva; semen; tears; lymphatic fluid; stool; bile; cerebrospinal fluid; and a mucus secretion.

7. The system of claim 6, wherein the liquid biopsy comprises whole blood, or a component thereof selected from serum or plasma.

8. The system of claim 1, wherein the adjustment to correct for the degree of nucleic acid shedding in the subject comprises identifying the amount of the first cfRNA present by correcting against a RNA organ Shedding Correction Factor (SCF) that is determined for the subject by performing an analysis of total cell free RNA (cf-RNA_{TOTAL}) in order to quantify an amount of mRNA present within the cfRNA_{TOTAL} that corresponds to each of two or more marker genes, wherein a marker gene is defined as a gene that is expressed principally and consistently in the organ/tissue; and determining the SCF as the mean concentration of mRNA of the each of two or more marker genes present within the cfRNA_{TOTAL}.

9. The system of claim 8, wherein the SCF is determined for the subject by isolating cfRNA_{TOTAL} from a liquid biopsy obtained from an individual subject, performing an analysis of the cfRNA_{TOTAL} in order to quantify an amount of two or more marker genes mRNAs present, designated as [cfRNA]_{Marker}, wherein a marker gene is defined as a gene that is expressed principally and consistently in the organ/tissue and at a high level; and determining the SCF according to the formula A:
$$SCF = 1 {0}^{6} \cdot {\sum }_{i = 1}^{N} {[ cfRNA ]}_{{Marker}_{i}} / ( N \times {[ cfRNA ]}_{TOTAL} ) \qquad A$$
where N is equal to the number of marker genes quantified.

10. The system of claim 1, wherein the cf-RNA encodes a cytochrome P450 monooxygenase (CYP) protein.

11. The system of claim 10, wherein CYP protein is selected from the group consisting of: Cytochrome P450 family 1 (CYP1); Cytochrome P450 family 2 (CYP2); Cytochrome P450 family 3 (CYP3); Cytochrome P450 family 4 (CYP4); Cytochrome P450 family 5 (CYP5); Cytochrome P450 family 7 (CYP7); Cytochrome P450 family 8 (CYP8); Cytochrome P450 family 11 (CYP11); Cytochrome P450 family 17 (CYP17); Cytochrome P450 family 19 (CYP19); Cytochrome P450 family 20 (CYP20); Cytochrome P450 family 21 (CYP21); Cytochrome P450 family 24 (CYP24); Cytochrome P450 family 26 (CYP26); Cytochrome P450 family 27 (CYP27); Cytochrome P450 family 39 (CYP39); Cytochrome P450 family 46 (CYP46); and Cytochrome P450 family 51 (CYP51).

12. The system of claim 11, wherein the CYP is selected from the group consisting of: CYP1A1; CYP1A2; CYP1B1; CYP2A6; CYP2A7; CYP2A13; CYP2B6; CYP2C8; CYP2C9; CYP2C18; CYP2C19; CYP2D6; CYP2E1; CYP3A4; CYP3A5; and CYP3A7.

13. The system of claim 1, wherein the cfRNA encodes a transferase selected from the group consisting of: a methyltransferase; a sulfotransferase; an N-acetyltransferase; and a glucuronosyltransferase.

14. The system of claim 13, wherein the transferase is selected from the group consisting of UGT1A1, UGT1A3, UGT1A4, UGT1A6, UGT1A9, UGT2B4, UGT2B7, UGT2B15 and UGT2B17; a glutathione-S-transferase; and a choline acetyl transferase.

15. The system of claim 1, wherein the cfRNA encodes a transporting protein selected from an ATP-binding cassette (ABC) transporter or a solute carrier (SLC) transporter.


SEQUENCE LISTING (2 sequences)

SEQ ID NO: 1 - DNA, 20 bp, Homo sapiens
  misc_feature (1)..(20): GADPH PRIMER
accacagtccatgccatcac

SEQ ID NO: 2 - DNA, 20 bp, Homo sapiens
  misc_feature (1)..(20): GADPH PRIMER
tccaccaccctgttgctgta